(19) United States
(12) Patent Application Publication
(10) Pub. No.: US 2016/0326582 A1
(43) Pub. Date: Nov. 10, 2016

(54) METHOD AND KIT FOR TARGET MOLECULE CHARACTERIZATION

(71) Applicant: Fry Laboratories, L.L.C., Scottsdale, AZ (US)

(72) Inventor: Jeremy Ellis, Mesa, AZ (US)

(21) Appl. No.: 15/146,794

(22) Filed: May 4, 2016

Related U.S. Application Data
(60) Provisional application No. 62/156,700, filed on May 4, 2015.

(51) Int. Cl. C12Q 1/68; G01N 21/64

(52) U.S. Cl. CPC C12Q 1/6874; G01N 21/6486; G01N 2021/6491

(57) ABSTRACT

Methods and kits for characterizing molecules, such as nucleic acids, large oligonucleotides, or portions thereof and/or to methods and kits for preparing samples for a characterization process are disclosed. Exemplary methods and kits can be used to form addressable portions, such that the addressable portions can be identified, sequenced, and reassembled to reconstruct an exemplary or characteristic molecule of a target molecule.

### CROSS-REFERENCE TO RELATED APPLICATIONS

This application claims the benefit of provisional Application Ser. No. 62/156,700, Entitled Method and Kit for Target Molecule Characterization, filed May 4, 2015, the contents of which are hereby incorporated herein by reference.

### INCORPORATION BY REFERENCE OF SEQUENCE LISTING

Incorporated by reference in its entirety herein is a computer-readable nucleotide/amino acid sequence listing submitted concurrently herewith and identified as follows: One 5 k byte ASCII (text) file named “6558800900_SequenceListing.txt” created on May 4, 2016, and is being submitted electronically via EFS-Web.

### FIELD OF INVENTION

The present disclosure generally relates to methods of characterizing molecular structures and to kits used for preparation of such characterization. More particularly, exemplary embodiments of the disclosure relate to methods of using one or more identifying molecules to obtain addressable portions, to methods of characterizing the target molecule based on the addressable portions, and to kits for producing the addressable portions.

### BACKGROUND OF THE DISCLOSURE

Molecular sequencing, such as DNA sequencing, can be used for a variety of applications, including identification or characterization of organisms, including microorganisms, disease diagnosis (e.g., detection of organisms that may cause a disease, identification of cancer cells, and/or detection of damaged chromosomes), as well as de novo identifying or characterizing previously unknown organisms.

Several techniques have been developed to sequence nucleic acids and oligonucleotides, such as portions of DNA strands. Early techniques generally relied on primer attachment, extensions of bases, and gel electrophoresis. Although these techniques were a significant improvement over prior techniques, such techniques were relatively expensive, slow, and a length of oligonucleotides that could be sequenced using these techniques was limited. Further, these techniques tended to be read-error prone. Next or second generation techniques were later developed. These second generation techniques are faster, more accurate (less read-error prone), and can take advantage of parallel processing. Further, several steps of next generation sequencing techniques can be automated.

When analyzing nucleic acids or large oligonucleotides—e.g., using second generation techniques—the nucleic acids or oligonucleotides are often cut or divided (e.g., using enzymes) into smaller pieces. The smaller pieces can then be amplified and then reassembled to obtain a sequence for the original or target molecule. Dividing the nucleic acids or large oligonucleotides into smaller pieces allows for faster and/or more accurate sequencing. However, errors can be introduced to the sequencing of the whole molecule during reassembly of the pieces.

Various second generation techniques can sequence relatively short nucleic acids or large oligonucleotides strands relatively accurately, while other techniques can sequence longer strands with relatively less accuracy. Thus, a tradeoff often exists between rapid sequencing techniques used to analyze divided pieces that are later reassembled and that can be error prone because of errors introduced during reassembly of the pieces and techniques that can be used to read longer nucleic acids or large oligonucleotides or portions thereof, but which are slower and/or may be prone to other forms of errors. Accordingly, improved systems, kits, and methods that allow for relatively fast and/or less error prone characterization of a target molecule by sequencing divided portions thereof are desired.

### SUMMARY OF THE DISCLOSURE

Exemplary embodiments of the present disclosure relate to methods and kits for characterizing molecules, such as nucleic acids, large oligonucleotides (e.g., 20 or more base pairs), or portions thereof and/or to methods and kits for preparing samples for a characterization process. As set forth in more detail below, exemplary methods and kits can be used to form addressable portions, such that the addressable portions can be identified, sequenced, and reassembled to reconstruct an exemplary or characteristic molecule of a target molecule. Because the exemplary methods sequence the smaller, addressable portions, the methods are suitable for use with next generation nucleotide sequencing. Furthermore, sequencing of the smaller, divided portions can proceed relatively quickly (e.g., in parallel), while using systems and/or techniques that produce relatively low read errors and/or sequence errors that can arise during reassembly of molecules.

In accordance with various exemplary embodiments of the disclosure, a method of characterizing a target molecule includes using one or more identifying molecules attached to the target molecule and forming two or more addressable divided sections. The one or more identifying molecules can include a random or unique identifying section. Employing the random or unique identifying section allows the divided sections to be addressed. Because the divided sections can be addressed, the two or more addressable divided sections can be used to reassemble a molecule that is characteristic of the target molecule. A representative target molecule can be constructed from a representative first portion and a corresponding representative second portion. Corresponding first and second sections can include the first and second identifying molecules or portions thereof. A representative first portion can be selected based on, for example, a most-common sequence or a most-common base at each base site within sequences of the first portions. Similarly, a representative second portion can be selected based on, for example, a most-common sequence or a most-common base at each base site within sequences of the second portions. Additionally or alternatively, higher-order sections can be used to characterize the target molecule.

In accordance with further exemplary embodiments of the disclosure, a method of characterizing a target molecule includes attaching a first identifying molecule to a first end of the target molecule, attaching a second identifying molecule to a second end of the target molecule, forming a plurality of continuous molecules including the first identifying molecule and the second identifying molecule; and dividing a first portion of the plurality of continuous molecules into first section(s) and a second portion of the plurality of continuous molecules into second section(s). Exemplary methods in accordance with these embodiments can further include steps of sequencing the first sections and sequencing the second sections. Various or selected first sections and second sections can then be reassembled, e.g., by identifying sections with corresponding first identifying molecules and second identifying molecules (or portions or products thereof) to reassemble (e.g., using a computer) a molecule characteristic or representing the target molecule. The first and second identifying molecules can include, for example, a primer and a random or unique tag. For example, the first identifying molecule can include a first or forward primer and a first tag. The second identifying molecule can include a second or reverse primer and a second tag. The first and second primers can be specific or random. The first and/or second tags can include one or more random sites (e.g., in the case of an oligonucleotide or nucleic acid, one or more sites in the tag molecule has an equal probability of including guanine (G), adenine (A), thymine (T), or cytosine (C) and/or an artificial base and/or a non-canonical base, such as one or more of: Inosine, Thiouridine, Uricil, Methyl-7-guanosine, Methylated RNA bases, RNA bases (if it were a hybrid molecule), Methylated DNA bases, Pseudouridine, Dihydrouridine, Dihydrouracil, Pseudouracil, Thiouracil, Methylcytosine, Methyl adenine, Isopentenyl adenine, Methyl guanidine, Queuosine, Wyosine, Diaminopurine, Isoguanine (isoC aka iso-dC), Isocytosine (isoG aka iso-dG), Diaminopyrimidine, Xanthine, Iosquinoline, Pyrrolo[2,3-b]pyridine, 2,4-difluorotoluene, 4-methylbenzimidazole, 2-amino-6-(2-thienyl)purine, pyrrole-2-carbaldehyde, 2,6-bis(ethylthiomethyl)pyridine (SPy and Ag ion), pyridine-2,6-dicarboxamide (Dipam), mondentate pyridine (Py) and Cu ion, 2′-deoxyinosine, Nitroazole-compounds, xDNA base pairs, yDNA base pairs, 2-amino-8-(2-thienyl)purine, pyridine-2-one, 7-(2-thienyl)imidazo[4,5-b]pyridine, pyrrole-2-carbaldehyde, 4-[3-(6-aminoheanamido)-1-propynyl]-2-nitropyrrole, 2-Aminopurine, 2,6-Diaminopurine (2-Amino-dA), 5-Bromo dU, deoxyuridine, Inverted dT, Inverted Dideoxy-T. By way of example, all sites in the tag molecule can be random. The step of dividing the portions of continuous molecules can include the use of additional identifying molecules. For example, a third and a fourth identifying molecule can be used to form the first section(s). The third identifying molecule can be selected to divide the continuous molecule near, adjacent, or within the first identifying molecule (e.g., to form a product with the first primer). For example, the third identifying molecule can be an inverse compliment to the first primer. The fourth identifying molecule can include a primer configured to divide the continuous molecule at a random or specific site. Similarly, to form the second section(s), a fifth identifying molecule can be selected to divide the continuous molecule near, adjacent, or within the second identifying molecule (e.g., to form a product with the first primer). The fifth identifying molecule can be, for example, an inverse compliment to the second primer. The sixth identifying molecule can include a primer configured to divide the continuous molecule at a random or specific site. In accordance with some examples of these embodiments, the fourth and sixth identifying molecules can be inverse compliments of each other (e.g., inverse compliment primers). A target molecule can be characterized by assembling corresponding first sections and section sections (e.g., using a computer). A representative first section can be selected based on, for example, a most-common first section; a longest first section; and/or by selecting most-common bases at one or more sites (e.g., all sites) within the first section. Similarly, a representative second section can be selected based on, for example, a most-common second section; a longest second section; and/or by selecting most-common bases at one or more sites (e.g., all sites) within the second section. Higher order sections can be formed by repeating the steps above on the first and/or second sections. The method can also include a step of attributing a quality score to the characterization of the target molecule, based on, for example, taking the highest quality score associated with each base in the plurality of sequences that constitute the most-common first or second sections as listed above. Exemplary methods can also include additional steps of diagnosing disease and/or determining risk factors using the methods described above.

In accordance with further exemplary embodiments of the disclosure, a method of characterizing a target molecule includes using a first amplification process (e.g., PCR or quantitative real-time PCR (qPCR)), attaching a first identifying molecule to a first end of the target molecule and attaching a second identifying molecule to a second end of the target molecule; forming a plurality of continuous molecules including the first identifying molecule and the second identifying molecule; and dividing a portion of the plurality of continuous molecules into a plurality of first sections and another portion of the plurality of continuous molecules into a plurality of second sections. The first identifying molecules can be the same or similar to those described above. The step of dividing can include a second amplification process (e.g., PCR or qPCR). Similar to the method above, the step of dividing can include the use of identifying molecules (e.g., primers), such as the third-sixth identifying molecules described above. Various (e.g., families of) first sections and second sections can be sequences and reassembled—e.g., using the techniques described above. A representative or characteristic target molecule can be characterized using the techniques described above—e.g., by selecting representative first, second, and/or higher order sections.

In accordance with further exemplary embodiments of the disclosure, a kit for characterizing a molecule includes a first identifying molecule, which can include a first primer and first tag attached to the first primer, and a second identifying molecule, which can include a second primer and a second tag attached to the second primer. Exemplary kits can also include one or more of a third, fourth, fifth, and/or sixth primer. Exemplary primers can be any first-sixth identifying molecule or primer described herein. The kit may include instructions and may utilize PCR or qPCR. The kit can also include a computer readable medium, having instructions thereon to perform the steps of determining representative first, second, or higher order sections, and/or reconstructing a target molecule as described herein—e.g., by selecting a representative from families of first sections and second sections.

In accordance with yet further exemplary embodiments of the disclosure, a method for preparing a sample for sequencing includes the steps of attaching a first identifying molecule to a first end of the target molecule, attaching a second identifying molecule to a second end of the target molecule, forming a plurality of continuous molecules including the first identifying molecule and the second identifying molecule, and dividing a first portion of the plurality of continuous molecules into first sections and a second portion of the plurality of continuous molecules into second sections. Exemplary methods in accordance with these embodiments can include additional steps noted above.

In accordance with yet additional exemplary embodiments of the disclosure, a method for preparing a sample for sequencing includes the steps of using a first amplification process, attaching a first identifying molecule to a first end of a target molecule and attaching a second identifying molecule to a second end of the target molecule, forming a plurality of continuous molecules including the first identifying molecule and the second identifying molecule, and dividing a first portion of the plurality of continuous molecules into first sections and a second portion of the plurality of continuous molecules into second sections.

Both the foregoing summary and the following detailed description are exemplary and explanatory only and are not restrictive of the present disclosure.

### BRIEF DESCRIPTION OF THE DRAWING FIGURES

A more complete understanding of exemplary embodiments of the present disclosure can be derived by referring to the detailed description and claims when considered in connection with the following illustrative figures.

FIG. 1 illustrates a molecule, including a target molecule, a first identifying molecule, and a second identifying molecule in accordance with exemplary embodiments of the disclosure.

FIG. 2 illustrates a target molecule having a first identifying molecule and a second identifying molecule attached thereto in accordance with exemplary embodiments of the disclosure.

FIG. 3 illustrates a continuous molecule and identifying molecules in accordance with further exemplary embodiments of the disclosure.

FIG. 4 illustrates another continuous molecule and identifying molecules in accordance with yet further exemplary embodiments of the disclosure.

FIG. 5 illustrates an exemplary first section in accordance with exemplary embodiments of the disclosure.

FIG. 6 illustrates an exemplary second section in accordance with yet further exemplary embodiments of the disclosure.

FIG. 7 illustrates another molecule, including a target molecule, a first identifying molecule, and a second identifying molecule in accordance with exemplary embodiments of the disclosure.

FIG. 8 illustrates a target molecule having a first identifying molecule and a second identifying molecule attached thereto in accordance with exemplary embodiments of the disclosure.

FIG. 9 illustrates a continuous molecule and identifying molecules in accordance with further exemplary embodiments of the disclosure.

FIG. 10 illustrates another continuous molecule and identifying molecules in accordance with yet further exemplary embodiments of the disclosure.

FIG. 11 illustrates an exemplary first section in accordance with exemplary embodiments of the disclosure.

FIG. 12 illustrates an exemplary second section in accordance with yet further exemplary embodiments of the disclosure.

FIG. 13 illustrates yet another molecule, including a target molecule, a first identifying molecule, and a second identifying molecule in accordance with exemplary embodiments of the disclosure.

FIG. 14 illustrates a target molecule having a first identifying molecule and a second identifying molecule attached thereto in accordance with exemplary embodiments of the disclosure.

FIG. 15 illustrates copies of a target molecule having a first identifying molecule and a second identifying molecule attached thereto in accordance with exemplary embodiments of the disclosure.

FIG. 16 illustrates a continuous molecule and identifying molecules in accordance with further exemplary embodiments of the disclosure.

FIG. 17 illustrates another continuous molecule and identifying molecules in accordance with yet further exemplary embodiments of the disclosure.

FIG. 18 illustrates an exemplary first section in accordance with exemplary embodiments of the disclosure.

FIG. 19 illustrates an exemplary second section in accordance with yet further exemplary embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve understanding of illustrated embodiments of the present disclosure.

### DETAILED DESCRIPTION OF EXEMPLARY EMBODIMENTS OF THE DISCLOSURE

The description of exemplary embodiments of methods and kits provided below is merely exemplary and is intended for purposes of illustration only; the following description is not intended to limit the scope of the disclosure. Moreover, recitation of multiple embodiments having stated features is not intended to exclude other embodiments having additional features or other embodiments incorporating different combinations of the stated features.

As set forth in more detail below, exemplary methods and kits can be used to characterize a target molecule, such a nucleic acid (or portion thereof) and/or oligonucleotides and/or prepare a sample for characterization of a target molecule. The nucleic acids can be naturally occurring or artificial (e.g., Iso-dC, Iso-dG, ZNA, etc.). The kits and methods can be used in conjunction with, for example, next generation sequencing systems to facilitate relatively fast and/or relatively accurate characterization of the target molecule.

Unless denoted otherwise, whenever an oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage. Nucleotides may also be identified as indicated below in Table 1.

TABLE 1
List of Nucleotide Abbreviations

| Symbol | Meaning | Origin of designation |
|---|---|---|
| A | A | adenine |
| G | G | guanine |
| C | C | cytosine |
| T | T | thymine |
| U | U | uracil |
| R | G or A | purine |
| Y | T/U or C | pyrimidine |
| M | A or C | amino |
| K | G or T/U | keto |
| S | G or C | strong interactions 3H- |
| | | bonds |
| W | A or T/U | weak interactions 2H- |
| | | bonds |
| B | G or C or T/U | not a |
| D | A or G or T/U | not c |
| H | A or C or T/U | not g |
| V | A or G or C | not t, not u |
| N | A or G or C or T/U, unknown, or other | any |

The majority of genes act by specifying polypeptide chains that form proteins. Proteins in turn make up living matter and catalyze all cellular processes.

Chemically, a genome is composed of deoxy-ribonucleic acid (“DNA”). Each DNA molecule is made up of repeating units of four nucleotide bases—adenine (“A”), thymine (“T”), cytosine (“C”), and guanine (“G”)—which are covalently linked, or bonded, together via a sugar-phosphate, or phosphodiester, backbone. DNA generally exists as two DNA strands intertwined as a double helix in which each base on a strand pairs, or hybridizes, with a complementary base on the other strand: A pairs with T, and C with G.

The linear order of nucleotide bases in a DNA molecule or oligonucleotide is referred to as its “sequence.” A sequence of such molecules thus denoted by a linear sequence of As, Ts, Gs, and Cs. “DNA sequencing” or “gene sequencing” refers to the process by which the linear order of nucleotides in a DNA segment or gene is determined. A gene's nucleotide sequence, in turn, encodes for a linear sequence of amino acids that comprise the protein encoded by the gene. Most genes have both “exon” and “intron” sequences. Exons are DNA segments that are necessary for the creation of a protein, i.e., that code for a protein. Introns are segments of DNA interspersed between the exons that, unlike exons, do not code for a protein.

The creation of a protein from a gene comprises two steps: transcription and translation. First, the gene sequence is “transcribed” into a different nucleic acid called ribonucleic acid (“RNA”). RNA has a chemically different sugar-phosphate backbone than DNA, and it utilizes the nucleotide base uracil (“U”) in place of thymine (“T”). For transcription, the DNA double helix is unwound and each nucleotide on the non-coding, or template, DNA strand is used to make a complementary RNA molecule of the coding DNA strand, i.e., adenine on the template DNA strand results in uracil in the RNA molecule, thymine results in adenine, guanine in cytosine, and cytosine in guanine. The resulting “pre-RNA,” like the DNA from which it was generated, contains both exon and intron sequences. Next, the introns are physically excised from the pre-RNA molecule, in a process called “splicing,” to produce a messenger RNA (“mRNA”).

Following transcription, the resulting mRNA is “translated” into the encoded protein. Genes, and their corresponding mRNAs, encode proteins via three-nucleotide combinations called codons. Each codon corresponds to one of the twenty amino acids that make up all proteins or a “stop” signal that terminates protein translation. For example, the codon adenine-thymine-guanine (ATG, or UTG in the corresponding mRNA), encodes the amino acid methionine. The relationship between the sixty-four possible codon sequences and their corresponding amino acids is known as the genetic code.

Changes, or mutations, in the sequence of a gene can alter the structure as well as the function of the resulting protein. Small-scale changes include point mutations in which a change to a single nucleotide alters a single amino acid in the encoded protein. For example, a base change in the codon GCU to CGU changes an alanine in the encoded protein to an arginine. Larger scale variations include the deletion, rearrangement, or duplication of larger DNA segments, ranging from several hundreds to over a million nucleotides, and result in the elimination, misplacement, or duplication of an entire gene or genes. While some mutations have little or no effect on processes, others result in disease or an increased risk of developing a particular disease. DNA (or oligonucleotide) sequencing can be used in clinical diagnostic testing to determine whether a gene contains mutations associated with a particular disease or risk of a particular disease.

Nearly every cell contains an entire genome. DNA in the cell, called “native” or “genomic” DNA, is packaged into chromosomes. Chromosomes are complex structures of a single DNA molecule wrapped around proteins called histones.

Genomic DNA can be extracted from its cellular environment using a number of well-established laboratory techniques. A particular segment of DNA, such as a gene, can then be excised or amplified from the DNA to obtain the isolated DNA segment of interest. DNA molecules can also be synthesized in the laboratory. One type of synthetic DNA molecule is complementary DNA (“cDNA”). cDNA is synthesized from mRNA using complementary base pairing in a manner analogous to RNA transcription. The process results in a double-stranded DNA molecule with a sequence corresponding to the sequence of an mRNA produced by the body. Because it is synthesized from mRNA, cDNA contains only the exon sequences, and thus none of the intron sequences, from a native gene sequence.

An oligonucleotide is a short segment of RNA or DNA, typically comprising approximately thirty or fewer nucleotide bases. Oligonucleotides may be formed by the cleavage or division of longer RNA/DNA segments, or may by synthesized by polymerizing individual nucleotide precursors, such as by polymerase chain reaction (PCR) and/or other known techniques. Automated synthesis techniques such as PCR may allow the synthesis of oligonucleotides up to 160 to 200 nucleotide bases. With respect to PCR, a “primer” allows DNA polymerase to extend an oligonucleotide and replicate the complementary strand. The length of an oligonucleotide is typically denoted in terms of “mer.” By way of non-limiting examples, an oligonucleotide having 25 nucleotide bases would be characterized as a 25-mer oligonucleotide. Because oligonucleotides readily bind to their respective complementary nucleotide, they may be used as probes for detecting particular DNA or RNA. The oligonucleotides can be made with standard molecular biology techniques known in the art and disclosed in manuals such as Sambrook et al., Molecular Cloning: A Laboratory Manual, Cold Spring Harbor Laboratory Press, Cold Spring Harbor, N.Y., USA (1989) or conventional nucleotide phosphoramidite chemistry and commercially available synthesizer instruments. The oligonucleotides can be DNA or RNA. Also contemplated are the RNA equivalents of the oligonucleotides and their complements.

The term “primer” refers to an isolated single stranded oligonucleotide sequence capable of acting as a point of initiation for synthesis of a primer extension product, which is complementary to the nucleic acid or nucleotide strand to be copied. A length and the sequence of the primer are generally such that they allow and/or are suitable to prime the synthesis of the extension products. A primer is typically about 5-50 nucleotides long, or from 10 to 40 nucleotides long. Specific length and sequence will depend on the complexity of the molecular (e.g., DNA or RNA) targets, as well as on the conditions of primer use, such as temperature and ionic strength.

As used herein, the terms “quantitative real time polymerase chain reaction,” “real-time polymerase chain reaction,” and “qPCR” are synonymous and refer to a laboratory technique based on a polymerase chain reaction used to amplify and simultaneously quantify a targeted DNA molecule. Frequently, real-time PCR is combined with reverse transcription to quantify messenger RNA and non-coding RNA in cells or tissues.

The oligonucleotides used as primers or probes may also comprise nucleotide analogues such as phosphorothiates, alkylphosphorothiates or peptide nucleic acids or may contain intercalating agents.

As most other variations or modifications introduced into the original DNA sequences, these variations may necessitate adaptions with respect to the conditions under which the oligonucleotide should be used to obtain the required specificity and sensitivity. However, the eventual results of hybridization will be essentially the same as those obtained with the unmodified oligonucleotides.

The introduction of these modifications may be advantageous in order to positively influence characteristics such as hybridization kinetics, reversibility of the hybrid-formation, biological stability of the oligonucleotide molecules, etc.

Two general exemplary PCR techniques can be used in connection with embodiments of the present disclosure. Both PCR assays described below have distinct advantages and disadvantages that differ from both a scientist and clinician perspective. The description below is for illustration purposes only; the disclosure is not limited to a particular form of PCR assay. The techniques are described below in connection with pan-genus analysis. The disclosure is not limited to such analysis.

First, traditional PCR-based detection methods utilize (e.g., specific) primers to amplify identifying sequences of an organism. Amplified products are visualized via gel electrophoresis, and bands that are within a certain size range can be further analyzed by restriction enzyme digest or by sequence analysis. This approach has significant advantages due to the flexibility in choices of primer design. Primers can be designed intentionally to amplify entire groups of related organisms and the stringency can be controlled by altering the primer positions, primer degeneracy, and primer annealing temperatures. Having the flexibility to make few assumptions about the target organism could provide detection of rare or novel species, thus providing immediate benefit to clinicians and their patients.

However, the above method is not without some weaknesses. Moderate stringency PCRs produce some potential positives that, upon sequence analysis, are identified as artifacts, which give the false appearance of a positive PCR signal. Therefore, effort spent on sequencing these false bands was wasted. Furthermore, the fact that this method intentionally is of moderate stringency opposes finely tuned and optimized detection levels. Other PCR-based methods could allow exceptionally sensitive detection down to even a single organismal genome in an entire patient sample. Thus far, it appears as if clinicians are willing to accept some loss of sensitivity for the increased chance at detecting rare or novel species.

Second, a newer PCR-based detection technique utilizes quantitative PCR (qPCR) that uses either fluorescently labeled nucleotides or probes to spectroscopically measure the levels of amplified product. This technique employs highly optimized and stringent probes, thus reducing the probability of pan-genus detection. However, qPCR is sensitive enough to detect exceptionally low copy numbers of the organismal genome.

The term “probe” refers to isolated single stranded sequence-specific oligonucleotides, which have a sequence that is complementary to the target sequence to be detected. Complementarity of the probe sequence to the target sequence is desired and complete for the central part of the probe (=core of the probe), where no mismatches to the target sequence are allowed. Towards the extremities (3′ or 5′) of the probe, minor variations in the probe sequence may sometimes occur, without affecting a species-specific hybridization behavior of the probe. The “core sequence” of the probe is the central part, and represents more than 70%, more than 80%, most often more than 90% of the total probe sequence.

Exemplary probes can specifically hybridize to nucleic acids for which they are designed. Exemplary probes can be represented from the 5′ end to the 3′ end—e.g., as single stranded DNA molecules. It should be understood, however, that these probes may also be used in their RNA form (wherein T is replaced by U), or in their complementary form.

Probes may be formed by cloning of recombinant plasmids containing inserts comprising the corresponding nucleotide sequences, if need be by cleaving the latter out from the cloned plasmids upon using the adequate nucleases and recovering them, e.g., by fractionation according to molecular weight. The probes can also be synthesized chemically, for instance, by the conventional phosphotriester method.

Some of the probes can have a length from about 10 to about 30 nucleotides. Variations are possible in the length of the probes and it should be clear that, since the central part of the probe is used for its hybridization characteristics, possible deviations of the probe sequence versus the target sequence may be allowable towards head and tail of the probe, especially when longer probe sequences are used. These variant probes can be evaluated experimentally in order to check if they result in equivalent hybridization characteristics compared to the original probes.

The term “isolated” as used herein means that the oligonucleotides disclosed herein are isolated from the environment in which they naturally occur. Isolated can be that the oligonucleotides are not a % more part of the genome of the respective species, and thus liberated from the remaining flanking nucleotides in the target region of the species. On the contrary, new (=heterologous) flanking regions may be added to the 3′ and/or 5′ end of the probe, in order to enhance its functionality. Functional characteristics, possibly provided by said heterologous flanking sequences, are, e.g., ease of attachment to a solid support, ease of synthesis, ease of purification, labeling function, etc. In a preferred form, the oligonucleotide is substantially free from other nucleic acid sequences, such as other chromosomal and extrachromosomal DNA and RNA, that normally accompany or interact with it as found in its naturally occurring environment. The term “isolated” oligonucleotide also embraces recombinant oligonucleotides and chemically synthesized oligonucleotides.

The term “complementary” nucleic acid as used herein means that the nucleic acid sequences can form a perfect base-paired double helix with each other.

The term “specific hybridization” refers to a selective hybridization of the probes disclosed herein to the nucleic acids of species to be detected (=target organism), and not to nucleic acids originating from strains belonging to other species (=non-target organisms). Specific hybridization in the context of the present disclosure also implies a selective hybridization of the disclosed probes to the target region of a species to be detected, and limits occasional “random” hybridization to other genomic sequences. Specificity is a feature which can be experimentally determined. Although it may sometimes be theoretically predictable, specificity refers to those non-target organisms which have been tested experimentally.

The term “sample,” as used herein, means anything designated for testing. The test sample is, or can be, derived from any biological source, such as, for example, blood, blood plasma, cell cultures, tissues and mosquito samples. The test sample can be used directly as obtained from the source, or following a pre-treatment to modify the character of the sample. Thus, the test sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, and purifying nucleic acid. A sample can include a clinical sample, such as a sample taken from blood, from the respiratory tract (sputum, bronchoalveolar lavage (BAL)), from cerebrospinal fluid (CSF), from the urogenital tract (vaginal secretions, urine), from the gastrointestinal tract (saliva, feces) or biopsies taken from organs, tissue, skin, teeth, bone, etc. The term sample can also refer to a sample of cultured cells, either cultured in liquid medium or on solid growth media. DNA present in said samples may be prepared or extracted according to any of the techniques known in the art. Alternatively, the sample could include synthetic material.

The “target” material (e.g., within a sample) can include, for example, genomic DNA or precursor ribosomal RNA of the organism to be detected (=target organism), portions thereof, or amplified versions thereof. These molecules are called target molecules or target nucleic acids.

A “tag” refers to a molecule that can be used to identify another molecule. For example, a tag can be used to identify families of first sections, second sections, and higher order sections. A tag can include one or more “random” sites. By way of examples, a tag can comprises an oligonucleotide where one or more base sites (or nucleotides) on the oligonucleotide are random—e.g., each site has an equal probability of including an A, G, C, or T base and/or an artificial base and/or a non-canonical base. A random tag can include an oligonucleotide having one or more random sites. The tag can include any suitable number of random sites. A tag can include one or more, two or more, three or more, four or more, or five or more bases, or 2 to about 50, 2 to about 20, or 2 to about 10 bases, of which any number of bases are random.

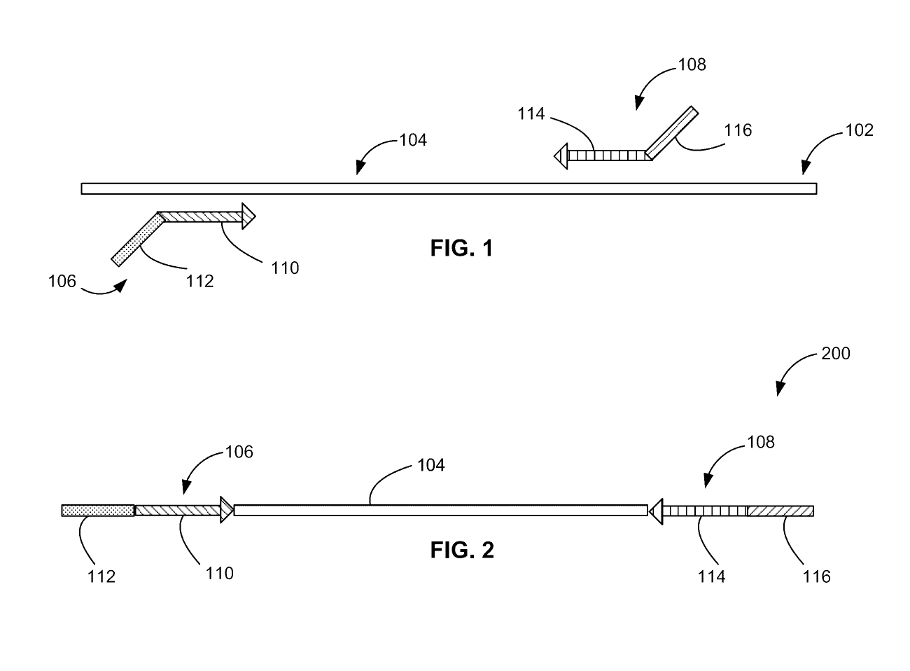

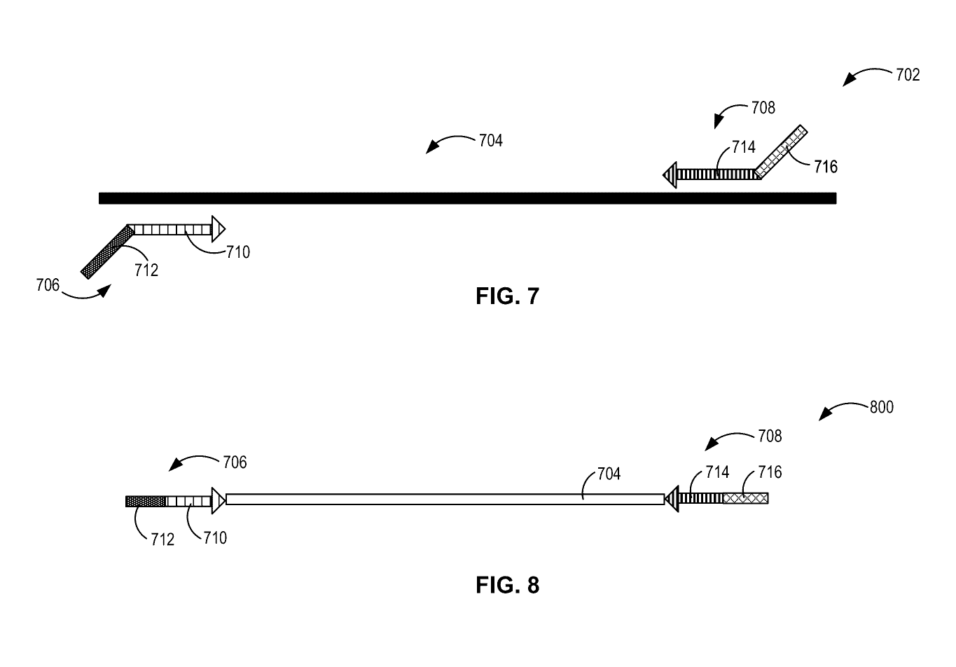

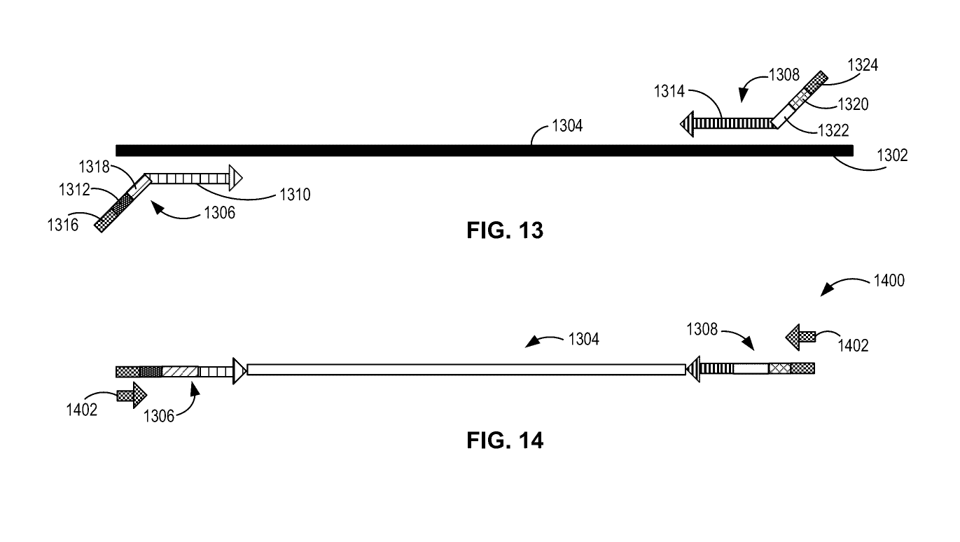

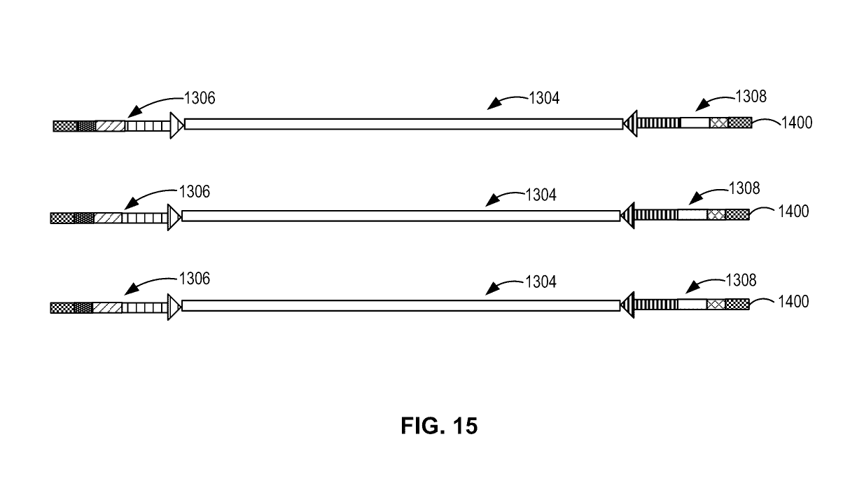

Turning now to the drawing figures, FIGS. 1-6 illustrate methods of characterizing a target molecule and methods for preparing a sample for sequencing in accordance with various exemplary embodiments of the disclosure. FIGS. 7-12 illustrate additional methods in accordance with the disclosure. And, FIGS. 13-19 illustrate yet additional methods. The methods described below are merely illustrative. Various steps and techniques described in connection with one set of examples can suitably be used in connection with methods described in connection with other examples, unless otherwise noted. For example, although adapters, spacers, or barcodes may be described in connection with some examples, such adapters, spacers, and/or barcodes can be used in any combination with other illustrative examples.

FIG. 1 illustrates a molecule 102, including a target region or target molecule 104, a first identifying molecule 106, and a second identifying molecule 108. In accordance with various embodiments of the disclosure, first and second identifying molecules 106, 108 are configured to attach to ends of target molecule 104 (e.g., the 3′ and 5′ ends of a nucleic acid). The first and second identifying molecules can include, for example, primers 110 (e.g., a first or forward primer) and 114 (e.g., a second or reverse primer). Primers 110 and/or 114 can be specific primers, designed to divide molecule 102 at specific locations, or primers 110 and/or 114 can be random primers, which are designed to divide molecule 102 at various—e.g., random—locations. By way of examples, primers 110 and 114 can be conserve region primers. In the illustrated example, first identifying molecule 106 further includes a first tag 112. Second identifying molecule includes a second tag 116. The first and second identifying tags can include unique or random molecules, such as a random oligonucleotide. Although not illustrated, the first and/or second identifying molecules can additionally include a spacer (described below) or known sequence between a primer and a tag. As discussed in more detail below, one or more identifying molecules (e.g., the first and/or second) can additionally or alternatively include a secondary priming site that can be suitable for random priming of the target molecule or of sections. The secondary priming site can allow for the introduction of a known priming site when a random priming site is necessarily unknown. This allows subsequent addressable division of first and second sections. In these cases, the spacer allows for differentiation between a single molecule tag (e.g., a combination of the first tag and the second tag) and the actual start of a priming site. The priming site could desirably be heavy G and C content to allow for proper annealing, but be short, e.g., less than about 20 or 30 nucleotides. The tags from either end in this situation wouldn't be directly linked (as described below); however, the tags would be contiguously read with only the secondary priming site spacing them out. They would still constitute the single molecule tag code that could allow for the reconstitution of the original sequence. As set forth in more detail below, the first and second identifying molecules can be used to address and identify corresponding sections (e.g., first and second and/or higher order sections) that can be used—e.g., virtually reassembled—to characterize (e.g., sequence) target molecule 104.

FIG. 2 illustrates identifying molecules 106 and 108 attached to target molecule 104. Identifying molecules 106, 108 can be attached using an amplification process, such as PCR. By way of example, about 5-10 PCR cycles can be used to produce molecules 200, which include target molecule 104 and identifying molecules 106 and 108.

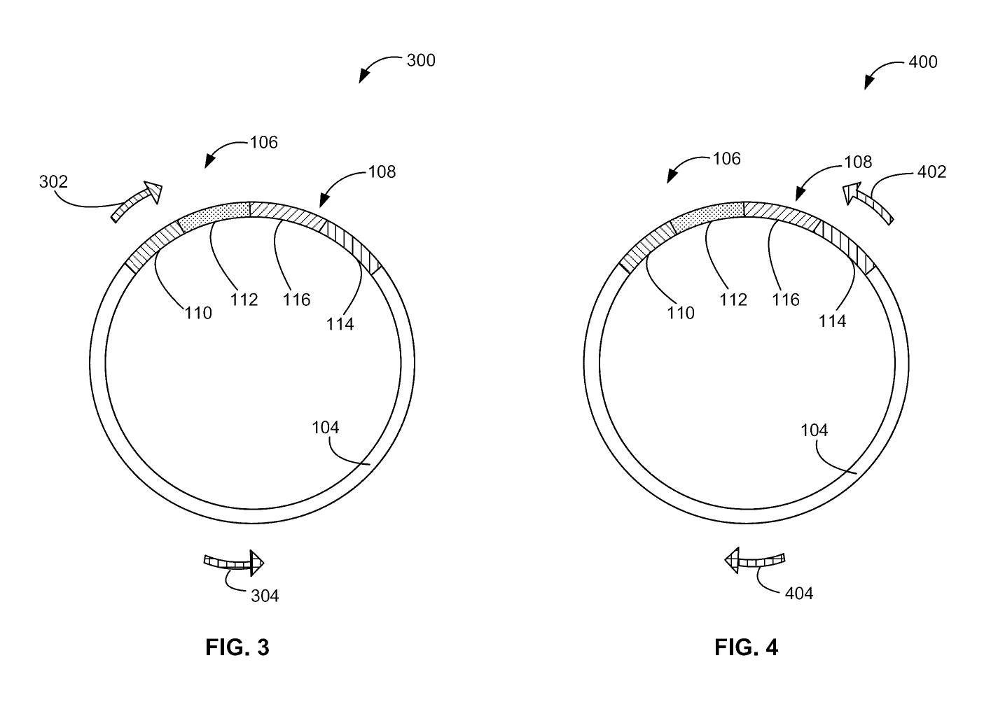

Once first and second identifying molecules 106 and 108 are attached to target molecule 104, a plurality of continuous molecules 300 and 400, illustrated in FIGS. 3 and 4, respectively, are formed. Continuous molecules 300, 400 can be formed by, for example, using one or more enzymes, such as DNA Ligase (e.g., E. coli ligase, thermostable bacterial ligases, T4 DNA ligase, DNA ligase I, III, IV, which all are from mammals, or Topoisomerase). In the illustrated example, first identifying molecule 106 and second identifying molecule 108 are linked together. Alternatively, another molecule can be used to couple first identifying molecule 106 and second identifying molecule 108. Such a molecule can be attached to a substrate or be available in solution. For example, a substrate that is addressable (has a tag) that can physically only link to a single molecule could be used. In accordance with yet further examples, the identifying molecules can be separated by a spacer, a barcode, or other molecule, which can initially form part of an identifying molecule or which can be added to a molecule from solution.

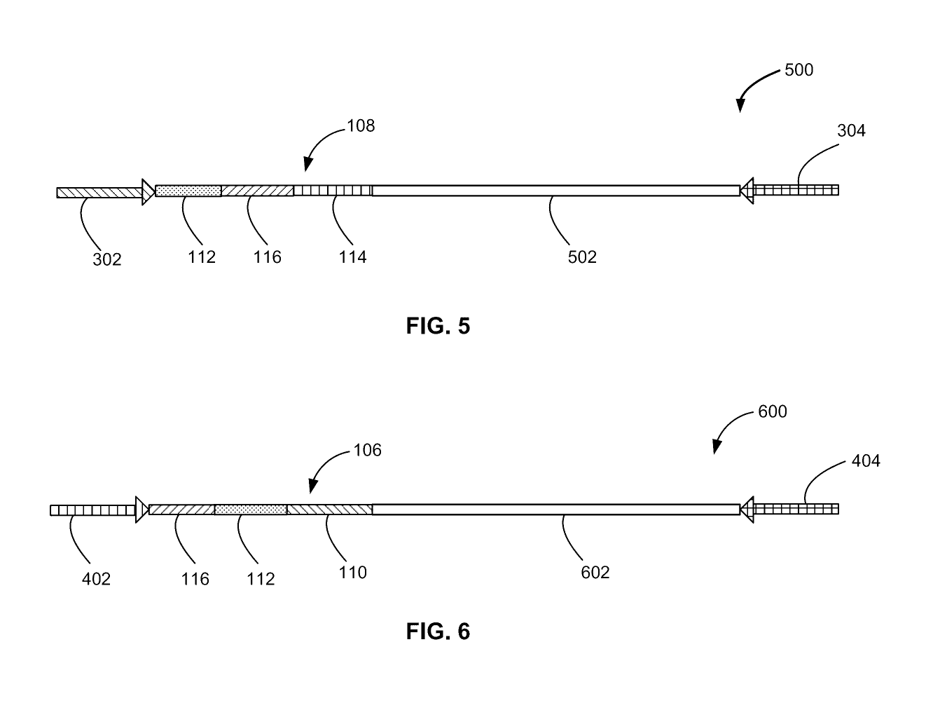

After continuous molecules 300, 400 are formed, the continuous molecules are divided into sections, such as first section 500, illustrated in FIG. 5, and second section 600, illustrated in FIG. 6. Sections 500 and 600 can then be sequenced or further divided into yet smaller sections—e.g., using the techniques described herein—and the smaller sections can be sequenced.

With reference to FIGS. 3 and 5, first section(s) 500 can be formed by using a third identifying molecule 302 and a fourth identifying molecule 304. Third and fourth identifying molecules 302, 304 can be or include primers designed to divide continuous molecule 300. Identifying molecules 302 and/or 304 can optionally include additional molecules, such as oligonucleotide, such as adapters, which can be sequence system dependent, spacers, barcodes, or the like. Identifying molecule 302 can include, for example, a forward primer configured to divide continuous molecule 300 at an edge of, near, or within identifying molecule 106 (such as at an end or near an end or within primer 110). In the illustrated example, identifying molecule 302 is an inverse compliment to primer 110, such that continuous molecule 300 is divided at or near first tag 112. However, primer 302 can be any suitable primer that forms a product with a portion of continuous molecule 300 to divide continuous molecule 300 at a desired location.

Fourth identifying molecule 304 can be configured to divide molecule 300 at various locations (a random primer) or at a specific location (a specific primer). For example, it may be desirable to divide molecule 300 approximately in half. In this case, fourth identifying molecule 304 can be designed or selected based on a length of molecule 300 and be configured to divide the molecule approximately in half.

As illustrated in FIG. 5, exemplary first section(s) 500 include third identifying molecule 302 (or a product of third identifying molecule 302 with a portion of continuous molecule 300), second identifying molecule 108 (including second primer 114 and second tag 116), first tag 112, a first section molecule 502, and fourth identifying molecule 304. Multiple first sections 500 can be formed from one or more continuous molecules 300 using a suitable amplification procedure.

With reference to FIGS. 4 and 6, second section(s) 600 can be formed by using a fifth identifying molecule 402 and a sixth identifying molecule 404. Fifth and Sixth identifying molecules 402, 404 can be or include primers designed to divide continuous molecule 400. Identifying molecules 402 and/or 404 can optionally include additional molecules, such as oligonucleotide, such as adapters, which can be sequence system dependent, spacers, barcodes, or the like. Identifying molecule 402 can include, for example, a forward primer configured to divide continuous molecule 400 at an edge of, near, or within identifying molecule 108 (such as at an end or near an end or within primer 114). In the illustrated example, identifying molecule 402 is an inverse compliment to primer 114, such that continuous molecule 400 is divided at or near second tag 116. However, primer 402 can be any suitable primer that forms a product with a portion of continuous molecule 400 to divide continuous molecule 400 at a desired location.

Sixth identifying molecule 404 can be configured to divide molecule 400 at various locations (a random primer) or at a specific location (a specific primer). For example, it may be desirable to divide molecule 400 approximately in half. In this case, sixth identifying molecule 404 can be designed or selected based on a length of molecule 400 and be configured to divide the molecule approximately in half.

As illustrated in FIG. 6, exemplary second section(s) 600 include first identifying molecule 106, second tag 116, fifth identifying molecule 402, a second section molecule 602, and sixth identifying molecule 404.

As noted above, first sections 500 and second sections 600 can be formed during an amplification process, such as PCR or qPCR. First sections 500 and second sections 600 can be formed during the same step or can be formed during sequential steps using the same or separate reaction vessels.

As noted above, once first sections 500 and second sections 600 are formed, the first and second sections can be sequenced. Alternatively, the steps set forth above can be repeated (where the first and second sections (or portions thereof) replace the target molecule), and the steps of forming sections are repeated until a desired number of steps have been performed or portion molecules are reduced to a desired size.

To characterize a target molecule, the first and second (and/or higher order) sections are sequenced. Corresponding sections can be identified by determining corresponding single molecule tags that include unique or random portions (e.g., tags 112 and 116). In the illustrated example, first portion 500 includes a single molecule tag including tags 112, 116 and second portion 600 includes the single molecule tag also including tags 116, 112. Exemplary or characteristic target molecules can be reconstructed by assembling corresponding first sections 500 and 600 with, e.g., first and second sections having the same single molecule tags.

A characteristic molecule can be determined using a variety of techniques. For example, a representative first segment (e.g., segment 500) can be selected from a family of first segments. In this context, “family” means first segments that are formed during amplification processes described herein. The representative first segment can be selected based on a most-common first segment found within the family of first segments, a first segment that has most-common bases at one or more (e.g., all) sites within the first segment or section of the target molecule (e.g., within section 502), the first segment having the longest length, or an artificially constructed first section based on the most-common bases at one or more sites along a sequence of the family of first sections, or other criteria. Similarly, a representative second segment (e.g., segment 600) can be selected from a family of second segments. The representative second segment can be selected based on a most-common second segment found within the family of second segments, a second segment that has most-common bases at one or more (e.g., all) sites within the second segment or section of the target molecule (e.g., within section 502), the second segment having the longest length, or an artificially constructed second section based on the most-common bases at one or more sites along a sequence of the family of second sections, or other criteria.

A computer-readable medium, having instructions stored thereon, can be used—e.g., as part of or in conjunction with a computer—to determine one or more representative first sections, second sections, and/or, if applicable, higher order sections. Corresponding sections are determined based on the single molecule tags as described herein. Using the computer and the computer-readable medium, corresponding sections can be used to virtually reassemble the sections and to form a characteristic target molecule. Further, the computer readable medium and/or the computer can be used to characterize sequence variations in the sections and/or target molecule. Such characterization can be used to sort out mutations, undesired oligonucleotides and/or nucleic acids, and/or erroneous reads.

Additional exemplary methods are illustrated in connection with FIGS. 7-12. The method steps described in connection with FIGS. 7-12 are similar to those described above in connection with FIGS. 1-6, except the methods described in connection with FIGS. 7-12 include the use of adapters that can be suitable for particular types of sequencing systems. Further, exemplary barcodes and spacers are illustrated.

With reference to FIG. 7, a molecule 702, including a target region or target molecule 704, a first identifying molecule 706, and a second identifying molecule 708, are illustrated. In accordance with various embodiments of the disclosure, first and second identifying molecules 706, 708 are configured to attach to ends of target molecule 104 (e.g., the 3′ and 5′ ends of a nucleic acid) and can be the same or similar to first and second identifying molecules 102, 104, described above. The first and second identifying molecules can include, for example, primers 710 (e.g., a first or forward primer) and 714 (e.g., a second or reverse primer), which can be specific or random primers. By way of examples, primers 710 and 714 are conserve region primers. In the illustrated example, first identifying molecule 706 further includes a first tag 712. Second identifying molecule 708 includes a second tag 716. The first and second identifying tags 712, 716 can include unique or random molecules, such as a random oligonucleotide. Although not illustrated, the first and/or second identifying molecules can additionally include a spacer (described below) or known sequence between a primer and a tag. As discussed in more detail below, one or more identifying molecules (e.g., the first and/or second) can additionally or alternatively include a secondary priming site that can be suitable for random priming of the target molecule or of sections. Table 2 below illustrates specific exemplary first and second identifying molecules suitable for use in characterizing variable regions 1,2 in 16S gene regions of bacteria.

TABLE 2

| Identifying | Exemplary | |
|---|---|---|
| Molecule | Tag | Exemplary Primer |
| First | NNNNN | AGAGTTTGATCCTGGCTCAG |
| | | (SEQ ID NO: 1) |
| Second | NNNNN | CTGCTGCCTYCCGTA |
| | | (SEQ ID NO: 2) |

FIG. 8 illustrates identifying molecules 706 and 708 attached to target molecule 704. Identifying molecules 706, 708 can be attached using an amplification process, such as PCR, as described above.

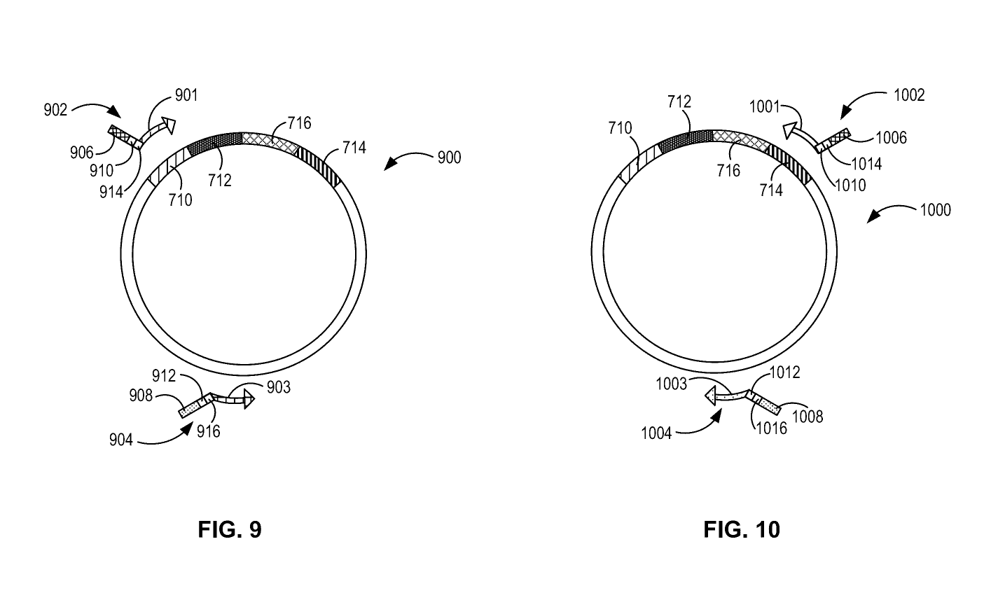

Once first and second identifying molecules 706 and 708 are attached to target molecule 704, a plurality of continuous molecules 900 and 1000, illustrated in FIGS. 9 and 10, respectively, are formed. Continuous molecules 900, 1000 can be formed by, for example, using the techniques described above.

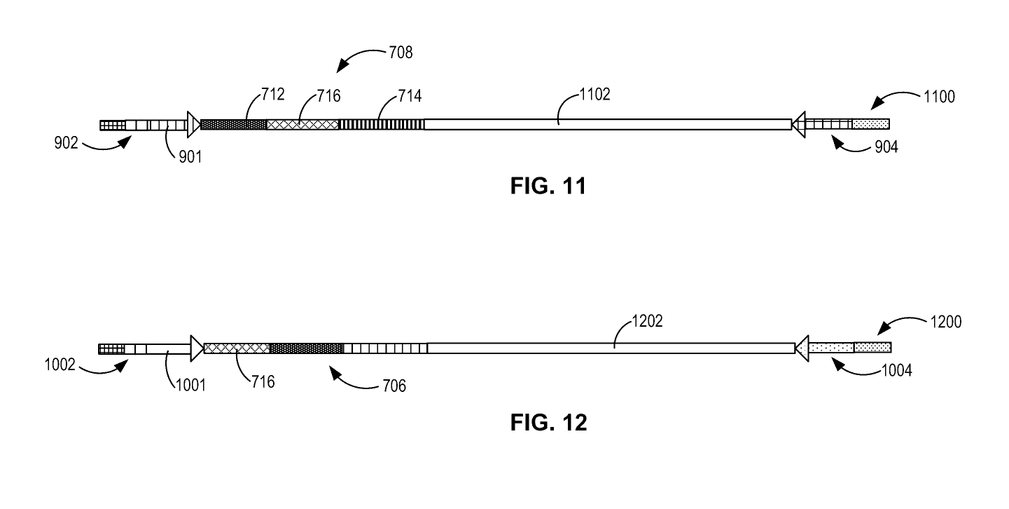

After continuous molecules 900, 1000 are formed, the continuous molecules are divided into sections, such as first section 1100, illustrated in FIG. 11, and second section 1200, illustrated in FIG. 12. Sections 1100 and 1200 can then be sequenced or further divided into yet smaller sections—e.g., using the techniques described herein—and the smaller sections can be sequenced.

With reference to FIGS. 9 and 11, first section(s) 1100 can be formed by using a third identifying molecule 902 and a fourth identifying molecule 904—e.g., using techniques described above. Third and fourth identifying molecules 902, 904 can be the same or similar to first and second molecules 106, 108 and can include primers 901, 903 designed to divide continuous molecule 900, and can optionally include additional molecules, such as oligonucleotide, such as adapters 906, 908, which can be sequence system dependent, spacers 910, 912, barcodes 912, 914, or the like.

Table 3 below illustrates specific exemplary third and fourth identifying molecules suitable for use in characterizing variable regions 1,2 in 16S gene regions of bacteria.

TABLE 3

| Identifying | | Exemplary | Exemplary | |
|---|---|---|---|---|
| Molecule | Exemplary Adapter | Barcode | Spacer | Exemplary Primer |
| Third | CCATCTCATCCCTGCGTGTCTCC | CAGATCCAT | GAT | CTGAGCCAGGATCAAAC |
| | GACTCAG (SEQ ID NO: 3) | C (SEQ ID | | TCT (SEQ ID NO: 5) |
| | | NO: 4) | | |
| Fourth | CCTCTCTATGGGCAGTCGGTGAT | | | TTACTCACCCGTNCGCCR |
| | (SEQ ID NO: 6) | | | CT (SEQ ID NO: 7) |

As illustrated in FIG. 11, exemplary first section(s) 1100 include third identifying molecule 902 (or a product of third identifying molecule 902 with a portion of continuous molecule 900), second identifying molecule 708 (including second primer 714 and second tag 716), first tag 712, a first section molecule 1102, and fourth identifying molecule 904. Multiple first sections 1100 can be formed from one or more continuous molecules 900 using a suitable amplification procedure.

With reference to FIGS. 10 and 12, second section(s) 1200 can be formed by using a fifth identifying molecule 1002 and a sixth identifying molecule 1004. Fifth and sixth identifying molecules 1002, 1004 can be or include primers designed to divide continuous molecule 1000, such as identifying molecules 402, 404 described above in connection with FIG. 4. Identifying molecules 1002 and/or 1004 can optionally include additional molecules, such as oligonucleotide, such as adapters 1006, 1008, which can be sequence system dependent, spacers 1010, 1012, barcodes 1014, 1016, or the like. Identifying molecule 1002 can include, for example, forward primer 1001 configured to divide continuous molecule 1000 at an edge of, near, or within identifying molecule 708 (such as at an end or near an end or within primer 714). In the illustrated example, primer 1001 is an inverse compliment to primer 714, such that continuous molecule 1000 is divided at or near second tag 716. However, primer 1001 can be any suitable primer that forms a product with a portion of continuous molecule 1000 to divide continuous molecule 1000 at a desired location.

Table 4 below illustrates specific exemplary fifth and sixth identifying molecules suitable for use in characterizing variable regions 1,2 in 16S gene regions of bacteria.

TABLE 4

| Identifying | | Exemplary | Exemplary | |
|---|---|---|---|---|
| Molecule | Exemplary Adapter | Barcode | Spacer | Exemplary Primer |
| Fifth | CCATCTCATCCCTGCGTGTCTCC | CAGATCCATC | GAT | TACGGRAGGCAGCAG |
| | GACTCAG (SEQ ID NO: 8) | (SEQ ID NO: 9) | | (SEQ ID NO: 10) |
| Sixth | CCTCTCTATGGGCAGTCGGTGA | | | TTACTCACCCGTNCGCCR |
| | T (SEQ ID NO: 11) | | | CT (SEQ ID NO: 12) |

As illustrated in FIG. 12, exemplary second section(s) 1200 include first identifying molecule 706, second tag 716, fifth identifying molecule 1002, a second section molecule 1202, and sixth identifying molecule 1004.

As noted above, first sections 1100 and second sections 1200 can be formed during an amplification process, such as PCR or qPCR. First sections 1100 and second sections 1200 can be formed during the same step or can be formed during sequential steps using the same or separate reaction vessels.

As further noted above, once first sections 1100 and second sections 1200 are formed, the first and second sections can be sequenced. Alternatively, the steps set forth above can be repeated (where the first and second sections (or portions thereof) replace the target molecule), and the steps of forming sections are repeated until a desired number of steps have been performed or portion molecules are reduced to a desired size.

Further exemplary methods are illustrated in connection with FIGS. 13-19. The method steps described in connection with FIGS. 13-19 are similar to those described above in connection with FIGS. 1-12, except the methods described in connection with FIGS. 13-19 include the use of random primers.

With reference to FIG. 13, a molecule 1302, including a target region or target molecule 1304, a first identifying molecule 1306, and a second identifying molecule 1308, are illustrated. In accordance with various embodiments of the disclosure, first and second identifying molecules 1306, 1308 are configured to attach to ends of target molecule 104 (e.g., the 3′ and 5′ ends of a nucleic acid) and can be the same or similar to first and second identifying molecules 102, 104, described above. The first and second identifying molecules can include, for example, primers 1310 (e.g., a first or forward primer) and 1314 (e.g., a second or reverse primer), which can be random primers. In the illustrated example, first identifying molecule 1306 further includes a first tag 1312, a first amplification priming site 1316, and a first artificial priming site 1318. Second identifying molecule 1308 includes a second tag 1320, a second amplification priming site 1324, and a second artificial priming site 1322. The first and second identifying tags 1312, 1320 can include unique or random molecules, such as a random oligonucleotide, as described above. Table 5 below illustrates specific exemplary first and second identifying molecules suitable for use in characterizing variable regions 1,2 in 16S gene regions of bacteria.

TABLE 5

| | Exemplary | | | |
|---|---|---|---|---|
| | Amplification | Exemplary | Exemplary Artificial | Exemplary |
| Identifying Molecule | Priming Site | Tag | Priming Site | Random Primer |
| First | GCTCGGACCGTGG | NNNNN | CCTGGGCCCTGGCCG | NNNNNNNNN |
| | (SEQ ID NO: 13) | | (SEQ ID NO: 14) | |
| Second | GCTCGGACCGTGG | NNNNN | GGCCACGGACCCGGC | NNNNNNNNN |
| | (SEQ ID NO: 15) | | (SEQ ID NO: 16) | |

FIG. 14 illustrates identifying molecules 1306 and 1308 attached to target molecule 1304. Identifying molecules 706, 708 can be attached using an amplification process, such as PCR. Additional copies of molecule 1400 can be formed using primers 1402. An exemplary primer is: GCTCGGACCGTGG (SEQ. ID NO:13)

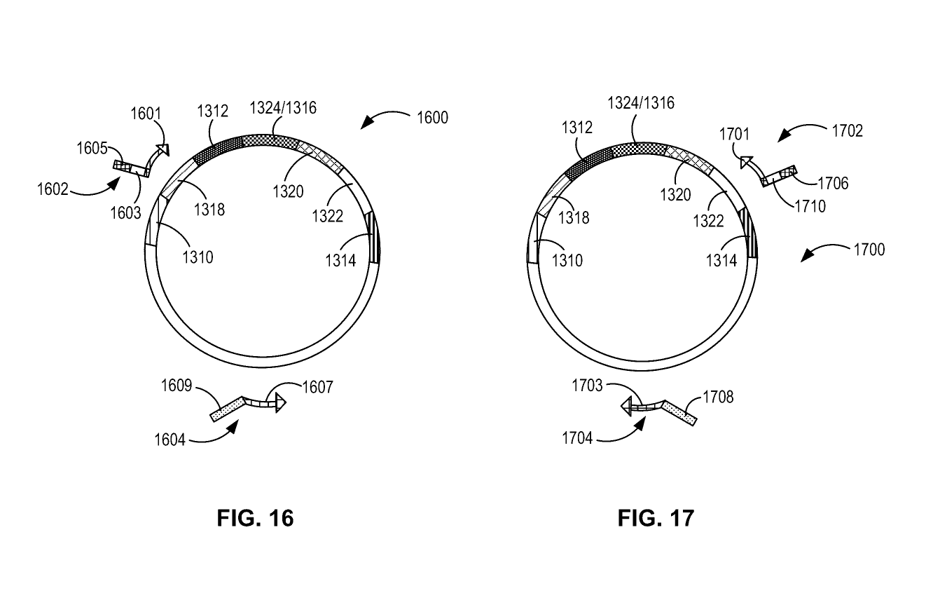

Once first and second identifying molecules 1306 and 1308 are attached to target molecule 1304, and molecule 1400 is amplified, a plurality of continuous molecules 1600 and 1700, illustrated in FIGS. 16 and 17, respectively, are formed. Continuous molecules 1600, 1700 can be formed by, for example, using the techniques described above.

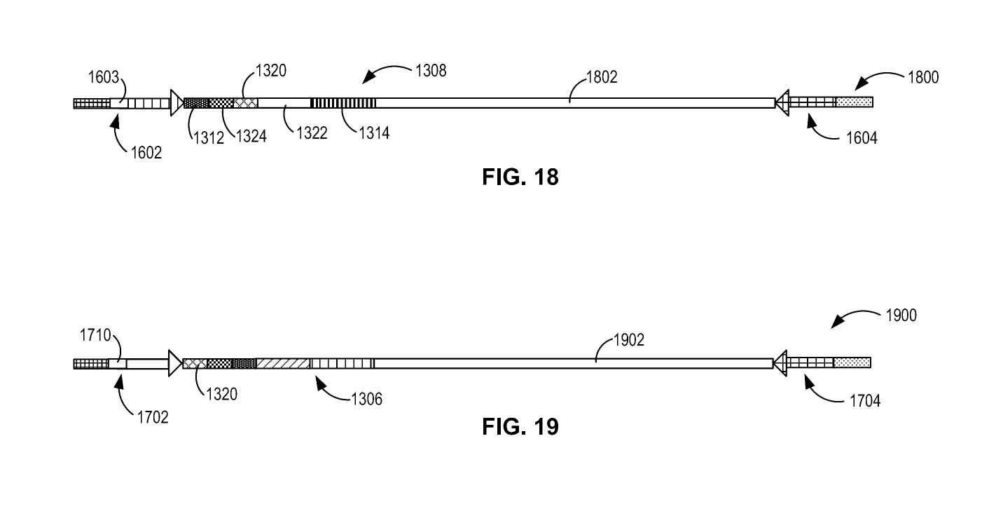

After continuous molecules 900, 1000 are formed, the continuous molecules are divided into sections, such as first section 1800, illustrated in FIG. 18, and second section 1900, illustrated in FIG. 19. Sections 1800 and 1900 can then be, and may be particularly well suited to be, sequenced or further divided into yet smaller sections—e.g., using the techniques described herein—and the smaller can then be sequenced.

With reference to FIGS. 16 and 18, first section(s) 1800 can be formed by using a third identifying molecule 1602 (e.g., including a primer 1601, a spacer 1603, and an adapter 1605) and a fourth identifying molecule 1604 (e.g., including a primer 1607 and an adapter 1609)—e.g., using techniques described above.

Table 6 below illustrates specific exemplary third and fourth (e.g., random) identifying molecules suitable for use in characterizing variable regions 1,2 in 16S gene regions of bacteria.

TABLE 6

| Identifying | | Exemplary | |
|---|---|---|---|
| Molecule | Exemplary Adapter | Spacer | Exemplary Primer |
| First | CCATCTCATCCCTGCGTGTCTCCGACTCAG | GAT | GCCGGGTCCGTGGCC |
| | (SEQ ID NO: 17) | | (SEQ ID NO: 18) |
| Second | CCTCTCTATGGGCAGTCGGTGAT | | NNNNNNNNNNNNNNN |
| | (SEQ ID NO: 19) | | (SEQ ID NO: 20) |

As illustrated in FIG. 18, exemplary first section(s) 1800 include third identifying molecule 1602 (or a product of third identifying molecule 1602 with a portion of continuous molecule 1600), second identifying molecule 1308 (including second primer 1314, second tag 1320, second artificial priming site 1322, and second amplification priming site 1324), a first section molecule 1802, first tag 1312, and fourth identifying molecule 1604. Multiple first sections 1800 can be formed from one or more continuous molecules 1600 using a suitable amplification procedure.

With reference to FIGS. 17 and 19, second section(s) 1900 can be formed by using a fifth identifying molecule 1702 and a sixth identifying molecule 1704. Fifth and sixth identifying molecules 1702, 1704 can be or include primers designed to divide continuous molecule 1700, such as identifying molecules 402, 404 described above in connection with FIG. 4. Identifying molecules 1702 and/or 1704 can optionally include additional molecules, such as oligonucleotide, such as adapters 1706, 1708, which can be sequence system dependent, spacers 1710, barcodes, or the like. Identifying molecule 1702 can include, for example, a forward primer 1701 configured to divide continuous molecule 1700 at an edge of, near, or within identifying molecule 1308 (such as at an end or near an end or within primer 1314). Similarly, sixth identifying molecule 1704 can include a primer 1703.

Table 7 below illustrates specific exemplary fifth and sixth identifying molecules suitable for use in characterizing variable regions 1,2 in 16S gene regions of bacteria.

TABLE 7

| Identifying | | Exemplary | |
|---|---|---|---|
| Molecule | Exemplary Adapter | Spacer | Exemplary Primer |
| First | CCATCTCATCCCTGCGTGTCTCCGACTCAG | GAT | GCCGGGTCCGTGGCC |
| | (SEQ ID NO: 17) | | (SEQ ID NO: 18) |
| Second | CCTCTCTATGGGCAGTCGGTGAT | | NNNNNNNNNNNNNNN |
| | (SEQ ID NO: 19) | | (SEQ ID NO: 20) |

As illustrated in FIG. 19, exemplary second section(s) 1900 include first identifying molecule 1306, second tag 1320, fifth identifying molecule 1702, a second section molecule 1902, and sixth identifying molecule 1704.

As noted above, first sections 1800 and second sections 1900 can be formed during an amplification process, such as PCR or qPCR. First sections 1800 and second sections 1900 can be formed during the same step or can be formed during sequential steps using the same or separate reaction vessels.

As further noted above, once first sections 1800 and second sections 1900 are formed, the first and second sections can be sequenced. Alternatively, the steps set forth above can be repeated (where the first and second sections (or portions thereof) replace the target molecule), and the steps of forming sections are repeated until a desired number of steps have been performed or portion molecules are reduced to a desired size. Target molecules can then be characterized as noted above.

### EXAMPLES

It is to be understood that various implementations may be utilized and compositional, as well as procedural, changes may be made without departing from the scope of this document. As a matter of convenience, various compositions and methods are described using exemplary materials, sizes, specifications, and the like. However, this document is not limited to the stated examples; other configurations are possible and within the teachings of the present disclosure.

The specific example provided below was used to characterize a molecule from the variable region 1/2 of Enterococcus faecalis. The reference number below refer to the example illustrated in FIGS. 7-12.
1. Amplify the target molecule through the first round PCR using Primer 1 (e.g., SEQ. ID NO:1) and Primer 2 (e.g., SEQ. ID NO:2) that incorporate tags, such as the tags illustrated in Table 2.

PCR Master Mix

| Component | Volume/Reaction |
|---|---|
| H2O | 3.48 μL |
| Enzyme/dNTP Concentrate | 5.00 μL |
| Primer 1 | 0.26 μL |
| Primer 2 | 0.26 μL |
| DNA Template | 1.00 μL |

### First Round PCR Conditions Example 1

| Step | Temperature (° C.) | Time | |
|---|---|---|---|
| 1 | 96 | 1:00 | min |
| 2 | 96 | 20 | sec |
| 3 | 42 | 30 | sec |
| 4 | 72 | 30 | sec |
| 5 | 9 repetitions of steps 2-4 | | |
| 6 | 72 | 5:00 | min |

| | | |
|---|---|---|
| 7 | 4 | indefinitely |

### First Round PCR Conditions Example 2

| Step | Temperature (° C.) | Time | |
|---|---|---|---|
| 1 | 96 | 2:00 | min |
| 2 | 96 | 20 | sec |
| 3 | 42* | 30 | sec |
| 4 | 72 | 30 | sec |
| 5 | 9 repetitions of steps 2-4 | | |
| 6 | 72 | 7:00 | min |

| | | |
|---|---|---|
| 7 | 4 | indefinitely |

*increased by 0.5° C. each round

2. Resulting PCR products are size selected (400-550 bp) by manual gel extraction or by automated size selection (Pippin Prep).
3. Size selected products are formed into continuous molecules using DNA ligase.

Ligation Reaction Mix

| Component | Volume/Reaction |
|---|---|
| Ligase | 1.0 μL |
| Size Selected DNA | 20.0 μL |

Incubate at 37° C. for 15 minutes.

4. Prepare two separate PCR amplification mixes using Primer 901 and Primer 903, also Primer 1001 and Primer 1003, as set forth in Tables 3 and 4.

Round 2 PCR Master Mix A

| Component | Volume/Reaction |
|---|---|
| H2O | 3.48 μL |
| Enzyme/dNTP Concentrate | 5.00 μL |
| Primer 901 | 0.26 μL |
| Primer 903 | 0.26 μL |
| DNA Template | 1.00 μL |

Round 2 PCR Master Mix B

| Component | Volume/Reaction |
|---|---|
| H2O | 3.48 μL |
| Enzyme/dNTP Concentrate | 5.00 μL |
| Primer 1001 | 0.26 μL |
| Primer 1003 | 0.26 μL |
| DNA Template | 1.00 μL |

Second Round Conditions PCR

| Step | Temperature (° C.) | Time | |
|---|---|---|---|
| 1 | 96 | 1:00 | min |
| 2 | 96 | 20 | sec |
| 3 | 50 | 30 | sec |
| 4 | 72 | 20 | sec |
| 5 | 24 repetitions of steps 2-4 | | |
| 6 | 72 | 5:00 | min |

| | | |
|---|---|---|
| 7 | 4 | indefinitely |

5. Resulting PCR products from both Mix A and Mix B are size selected (150-350 bp) by manual gel extraction or by automated size selection (Pippin Prep).
6. Templates are prepared, mixed in equimolar ratios, and sequenced as normal using the IonTorrent PGM protocols.
7. Resulting data is processed using software described above. Corresponding first sections and second sections were reassembled to characterize the target molecule.

Kits according to various embodiments of the disclosure include one or more reagents—e.g., reagents useful for practicing one or more methods of the disclosure. A kit can include a package with one or more containers holding the reagent(s) (e.g., primers and/or probe(s)), as one or more separate compositions or, optionally, as admixture where the compatibility of the reagents will allow. A kit can also include other material(s) that may be desirable from a user standpoint, such as a buffer(s), a diluent(s), a standard(s), and/or any other material useful in sample processing, washing, or conducting any other step of the assay.

Kits according to the disclosure can include instructions for carrying out one or more of the methods of the disclosure. Instructions included in kits of the disclosure can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

By way of examples, exemplary kits contain one or more identifying molecules, such as the identifying molecules described above. The kits can be used to characterize a molecule as noted above. For example, a kit can include a first identifying molecule, including a first primer and first tag attached to the first primer, and a second identifying molecule, including a second primer and a second tag attached to the second primer. Exemplary kits can further include one or more of third, fourth, fifth, and/or sixth primers as discussed above, and/or additional primers that can be used for, for example, further dividing the first sections and/or second sections. Additionally and/or alternatively, exemplary kits can include a computer readable medium, having instructions thereon to perform various steps described herein, such as the steps of identifying first, second, and/or other sections, sorting out undesired sequences, and/or reconstructing the target molecule.

The kit can further include the four deoxynucleotide phosphates (dATP, dGTP, dCTP, dTTP) and/or an effective amount of a nucleic acid polymerizing enzyme. A number of enzymes are known in the art which are useful as polymerizing agents. These include, but are not limited to, E. coli DNA polymerase I, Klenow fragment, bacteriophage T7 RNA polymerase, reverse transcriptase, and polymerases derived from thermophilic bacteria, such as Thermus aquaticus. The latter polymerases are known for their high temperature stability, and include, for example, the Taq DNA polymerase I. Other enzymes, such as Ribonuclease H, can be included in the assay kit for regenerating the template DNA. Other optional additional components of the kit include, for example, means used to label the probe and/or primer (such as a fluorophore, quencher, chromogen, etc.), and the appropriate buffers for reverse transcription, PCR, or hybridization reactions.

In accordance with various embodiments of the disclosure, probes can be used to facilitate sequencing. Nucleic acids, including oligonucleotide probes, in the methods and compositions described herein can be labeled with a reporter. A reporter is a molecule that facilitates the detection of a molecule to which it is attached. Numerous reporter molecules that may be used to label nucleic acids are known. Direct reporter molecules include fluorophores, chromophores, and radiophores. Non-limiting examples of fluorophores include a red fluorescent squarine dye, such as 2,4-Bis[1,3,3-trimethyl-2-indolinylidenemethyl]cyclobutenediylium-1,3-dioxolate, an infrared dye, such as 2,4Bis[3,3-dimethyl-2-(1H-benz[e]indolinylidenemethyl)]cyclobutenediylium-1,3-dioxolate, or an orange fluorescent squarine dye, such as 2,4-Bis[3,5-dimethyl-2-pyrrolyl]cyclobutenediylium-1,3-diololate. Additional non-limiting examples of fluorophores include quantum dots, Alexa Fluor® dyes, AMCA, BODIPY® 630/650, BODIPY® 650/665, BODIPY®-FL, BODIPY®-R6G, BODIPY®-TMR, BODIPY® TRX, Cascade Blue®, CyDye™, including, but not limited, to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™ TAMRA™, TET™, Tetramethylrhodamine, or Texas Red®. A signal amplification reagent, such as tyramide (PerkinElmer), may be used to enhance the fluorescence signal. Indirect reporter molecules include biotin, which must be bound to another molecule such as streptavidin-phycoerythrin for detection. In a multiplex reaction, the reporter attached to the primer or the dNTP may be the same for all reactions in the multiplex reaction if the identities of the amplification products can be determined based on the specific location or identity of the solid support to which they hybridize.

It is also contemplated that fluorophore/quencher-based detection systems may be used with the methods and compositions disclosed herein. When a quencher and fluorophore are in proximity to each other, the quencher quenches the signal produced by the fluorophore. A conformational change in the nucleic acid molecule separates the fluorophore and quencher to allow the fluorophore to emit a fluorescent signal. Fluorophore/quencher-based detection systems reduce background and therefore allow for higher multiplexing of primer sets compared to free-floating fluorophore methods, particularly in closed tube and real-time detection systems.

In particular embodiments, molecules useful as quenchers include, but are not limited to, tetramethylrhodamine (TAMRA), DABCYL (DABSYL, DABMI or methyl red) anthroquinone, nitrothiazole, nitroimidazole, malachite green, Black Hole Quenchers®, e.g., BHQ1 (Biosearch Technologies), Iowa Black® or ZEN quenchers (from Integrated DNA Technologies, Inc.) (e.g., 3′ Iowa Black® RQ-Sp aka 3IABRQSp and 3′ Iowa Black® FQ aka 3IABkFQ), TIDE Quencher 2 (TQ2) and TIDE Quencher 3 (TQ3) (from AAT Bioquest).

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.); Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Preferably, commercially available linking moieties are employed that can be attached to an oligonucleotide during synthesis, e.g., available from Integrated DNA Technologies (Coralville, Iowa) or Eurofins MWG Operon (Huntsville, Ala.).

Rhodamine and fluorescein dyes are also conveniently attached to the 5′ hydroxyl of an oligonucleotide at the conclusion of solid-phase synthesis by way of dyes derivatized with a phosphoramidite moiety, e.g., Woo et al., U.S. Pat. No. 5,231,191; and Hobbs, Jr., U.S. Pat. No. 4,997,928.

The amplifying steps described herein can be performed using, for example, any type of nucleic acid template-based method, such as PCR technology. PCR is a technique widely used in molecular biology to amplify a piece of DNA by in vitro enzymatic replication. Typically, PCR applications employ a heat-stable DNA polymerase, such as Taq polymerase. This DNA polymerase enzymatically assembles a new DNA strand from nucleotides (dNTPs) using single-stranded DNA as a template and DNA primers to initiate DNA synthesis. A basic PCR reaction uses several components and reagents including: a DNA template that contains the target sequence to be amplified; one or more primers, which are complementary to the DNA regions at the 5′ and 3′ ends of the target sequence; a DNA polymerase (e.g., Taq polymerase) that preferably has a temperature optimum at around 70° C.; deoxynucleotide triphosphates (dNTPs); a buffer solution providing a suitable chemical environment for optimum activity and stability of the DNA polymerase; divalent cations, typically magnesium ions (Mg2+); and monovalent cation potassium ions.

PCR technology uses thermal strand separation followed by thermal dissociation. During this process, at least one primer per strand, cycling equipment, high reaction temperatures and specific thermostable enzymes are used (see, e.g., U.S. Pat. Nos. 4,683,195 and 4,883,202). Alternatively, it is possible to amplify the DNA at a constant temperature (Nucleic Acids Sequence Based Amplification (NASBA) Kievits, T., et al., J. Virol Methods, 1991; 35, 273-286; and Malek, L. T., U.S. Pat. No. 5,130,238; T7 RNA polymerase-mediated amplification (TMA)) (Giachetti C, et al., J Clin Microbiol 2002 July; 40(7):2408-19; or Strand Displacement Amplification (SDA), Walker, G. T. and Schram, J. L., European Patent Application Publication No. 0 500 224 A2; Walker, G. T., et al., Nuc. Acids Res., 1992; 20, 1691-1696).

Thermal cycling subjects the PCR sample to a defined series of temperature steps. Each cycle typically has 2 or 3 discrete temperature steps. The cycling is often preceded by a single temperature step (“initiation”) at a high temperature (>90° C.), and followed by one or two temperature steps at the end for final product extension (“final extension”) or brief storage (“final hold”). The temperatures used and the length of time they are applied in each cycle depend on a variety of parameters. These include the enzyme used for DNA synthesis, the concentration of divalent ions and dNTPs in the reaction, and the melting temperature (Tm) of the primers. Commonly used temperatures for the various steps in PCR methods are: initialization step—94-96° C.; denaturation step—94-98° C.; annealing step—50-65° C.; extension/elongation step—70-74° C.; final elongation—70-74° C.; final hold—4-10° C.

As noted above, real-time polymerase chain reaction, also called quantitative real time polymerase chain reaction (QRT-PCR) or kinetic polymerase chain reaction, is used to amplify and simultaneously quantify a targeted DNA molecule. It enables both detection and quantification (as absolute number of copies or relative amount when normalized to DNA input or additional normalizing genes) of a specific sequence in a DNA sample. Real-time PCR may be combined with reverse transcription polymerase chain reaction to quantify low abundance RNAs. Relative concentrations of DNA present during the exponential phase of real-time PCR are determined by plotting fluorescence against cycle number on a logarithmic scale. Amounts of DNA may then be determined by comparing the results to a standard curve produced by real-time PCR of serial dilutions of a known amount of DNA.

Multiplex-PCR and multiplex real-time PCR use of multiple, unique primer sets within a single PCR reaction to produce amplicons of different DNA sequences. By targeting multiple genes at once, additional information may be gained from a single test run that otherwise would require several times the reagents and more time to perform. Annealing temperatures for each of the primer sets should be optimized to work within a single reaction.

Multiplex-PCR and multiplex real-time PCR may also use unique sets or pools of oligonucleotide probes to detect multiple amplicons at once. In some embodiments, a method described herein can be used with multiplex quantitative real time PCR (qPCR) with unique pools of oligonucleotide probes.

The methods disclosed herein may also utilize asymmetric priming techniques during the PCR process, which may enhance the binding of the reporter probes to complimentary target sequences. Asymmetric PCR is carried with an excess of the primer for the chosen strand to preferentially amplify one strand of the DNA template more than the other.

Amplified nucleic acid can be detected using a variety of detection technologies well known in the art. For example, amplification products may be detected using agarose gel by performing electrophoresis with visualization by ethidium bromide staining and exposure to ultraviolet (UV) light, by sequence analysis of the amplification product for confirmation, or hybridization with an oligonucleotide probe.

The oligonucleotide probe may comprise a flourophore and/or a quencher. The oligonucleotide probe may also contain a detectable label including any molecule or moiety having a property or characteristic that is capable of detection, such as, for example, radioisotopes, fluorophores, chemiluminophores, enzymes, colloidal particles, and fluorescent microparticles.

Probe sequences can be employed using a variety of methodologies to detect amplification products. Generally, all such methods employ a step where the probe hybridizes to a strand of an amplification product to form an amplification product/probe hybrid. The hybrid can then be detected using labels on the primer, probe or both the primer and probe. Examples of homogeneous detection platforms for detecting amplification products include the use of FRET (fluorescence resonance energy transfer) labels attached to probes that emit a signal in the presence of the target sequence. “TaqMan” assays described in U.S. Pat. Nos. 5,210,015; 5,804,375; 5,487,792 and 6,214,979 and Molecular Beacon assays described in U.S. Pat. No. 5,925,517 are examples of techniques that can be employed to detect nucleic acid sequences. With the “TaqMan” assay format, products of the amplification reaction can be detected as they are formed or in a so-called “real time” manner. As a result, amplification product/probe hybrids are formed and detected while the reaction mixture is under amplification conditions.

For example, the PCR probes may be TaqMan® probes that are labeled at the 5′ end with a fluorophore and at the 3′ end with a quencher molecule. Suitable fluorophores and quenchers for use with TaqMan® probes are disclosed in U.S. Pat. Nos. 5,210,015, 5,804,375, 5,487,792 and 6,214,979 and WO 01/86001 (Biosearch Technologies). Quenchers may be Black Hole Quenchers disclosed in WO 01/86001.

Nucleic acid hybridization can be done using techniques and conditions known in the art. Specific hybridization conditions will depend on the type of assay in which hybridization is used. Hybridization techniques and conditions can be found, for example, in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York) and Sambrook et al. (1989) Molecular Cloning. A Laboratory Manual (2d ed., Cold Spring Harbor Laboratory Press, Plainview, N.Y.).

Hybridization of nucleic acid may be carried out under stringent conditions. “Stringent conditions” or “stringent hybridization conditions” can mean conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified. Alternatively, stringency conditions can be adjusted to allow some mismatching in sequences so that lower degrees of similarity are detected.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents, such as formamide. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours, or less depending on the assay format.

It should be noted that the oligonucleotides of this disclosure can be used as primers or probes, depending on the intended use or assay format. For example, an oligonucleotide used as a primer in one assay can be used as a probe in another assay. The grouping of the oligonucleotides into primer pairs and primer/probe sets reflects certain implementations only. However, the use of other primer pairs comprised of forward and reverse primers selected from different preferred primer pairs is specifically contemplated.

Quantitative Real-Time PCR (qPCR) Detection Chemistries

There are several commercially available nucleic acid detection chemistries currently used in qPCR. These chemistries include DNA binding agents, FRET-based nucleic acid detection, hybridization probes, molecular beacons, hydrolysis probes, and dye-primer based systems. Each of these chemistries is discussed in more detail below.

DNA Binding Agents

The first analysis of kinetic PCR was performed by Higuchi et al. who used ethidium bromide to bind double-stranded DNA products (Higuchi et al., 1992; Higuchi et al., 1993; U.S. Pat. No. 5,994,056; U.S. Published Application No. 2001/6171785). Ethidium bromide, like other DNA binding agents used in kinetic PCR, is able to increase in fluorescent intensity upon binding. The resulting increase in signal can be recorded over the course of the reaction, and plotted versus the cycle number. Recording the data in this way is more indicative of the initial concentration of the sample of interest compared to end-point analysis.

Binding dyes are relatively inexpensive as compared to other detection chemistries. The advantages of using these binding dyes are their low cost and excellent signal-to-noise ratios. Disadvantages include their non-specific binding properties to any double-stranded DNA in the PCR reaction, including amplicons created by primer-dimer formations (Wittwer et al., 1997). In order to confirm the production of a specific amplicon, a melting curve analysis should be performed (Ishiguro et al., 1995). Another drawback is that amplification of a longer product will generate more signal than a shorter one. If amplification efficiencies are different, quantification may be even more inaccurate (Bustin and Nolan, 2004).

SYBR® Green I from Invitrogen™ (Carlsbad, Calif.) is a popular intercalating dye (Bengtsson et al., 2003). SYBR® Green I is a cyclically substituted asymmetric cyanine dye (Zipper et al., 2004; U.S. Pat. No. 5,436,134; U.S. Pat. No. 5,658,751). A minor groove-binding asymmetric cyanine dye known as BEBO has been used in real-time PCR. BEBO causes a non-specific increase in fluorescence with time, perhaps due to a slow aggregation process, and is less sensitive compared to SYBR® Green I. A similar dye called BOXTO has also been reported for use in qPCR (Bengtsson et al., 2003; U.S. Published Application No. 2006/0211028). Like BEBO, BOXTO is less sensitive than SYBR® Green I (U.S. Published Application No. 2006/0211028).

Other common reporters include YO-PRO-1 and thiazole orange (TO), which are intercalating asymmetric cyanine dyes (Nygren et al., 1998). While these dyes exhibit large increases in fluorescence intensity upon binding, TO and Oxazole Yellow (YO) have been reported to perform poorly in real-time PCR (Bengtsson et al., 2003). Other dyes that may be used include, but are not limited to, pico green, acridinium orange, and chromomycin A3 (U.S. Published Application No. 2003/6569627). Dyes that may be compatible with real-time PCR can be obtained from various vendors, such as Invitrogen, Cambrex Bio Science (Walkersville, Md.), Rockland Inc. (Rockland, Me.), Aldrich Chemical Co. (Milwaukee, Wis.), Biotium (Hayward, Calif.), TATAA Biocenter AB. (Goteborg, Sweden) and Idaho Technology (Salt Lake City, Utah) (U.S. Published Application No. 2007/0020672).

A dye known as EvaGreen™ (Biotium) has shown promise in that it is designed to not inhibit PCR, and is more stable in alkaline conditions as compared to SYBR® Green I (Dorak, 2006; U.S. Published Application No. 2006/0211028). Other newer dyes include the LCGreen® dye family (Idaho Technology). LCGreen® I and LCGreen® Plus are the most commercially competitive of these dyes. LCGreen® Plus is considerably brighter than LCGreen® (U.S. Published Application No. 2007/0020672; Dorak, 2006; U.S. Published Application No. 2005/0233335; U.S. Published Application No. 2066/0019253).

FRET-Based Nucleic Acid Detection

Many real-time nucleic acid detection methods utilize labels that interact by Förster Resonance Energy Transfer (FRET). This mechanism involves a donor and acceptor pair wherein the donor molecule is excited at a particular wavelength, and subsequently transfers its energy non-radiatively to the acceptor molecule. This typically results in a signal change that is indicative of the proximity of the donor and acceptor molecules to one another.

Early methods of FRET-based nucleic acid detection that lay a foundation for this technology, in general, include work by Heller et al. (U.S. Pat. Nos. 4,996,143; 5,532,129; and 5,565,322). These patents introduce FRET-based nucleic acid detection by including two labeled probes that hybridize to the target sequence in close proximity to each other. This hybridization event causes a transfer of energy to produce a measurable change in spectral response, which indirectly signals the presence of the target.

Cardullo et al. established that fluorescence modulation and nonradiative fluorescence resonance energy transfer can detect nucleic acid hybridization in solution (Cardullo et al., 1988). This study used three FRET-based nucleic acid detection strategies. The first includes two 5′ labeled probes that were complementary to one another, allowing transfer to occur between a donor and acceptor fluorophore over the length of the hybridized complex. In the second method, fluorescent molecules were covalently attached to two nucleic acids, one at the 3′ end and the other at the 5′ end. The fluorophore-labeled nucleic acids hybridized to distinct but closely-spaced sequences of a longer, unlabeled nucleic acid. Finally, an intercalating dye was used as a donor for an acceptor fluorophore that was covalently attached at the 5′ end of the probe.

Morrison et al. (1989) used complementary-labeled probes to detect unlabeled target DNA by competitive hybridization, producing fluorescence signals which increased with increasing target DNA concentration. In this instance, two probes were used that were complementary to one another and labeled at their 5′ and 3′ ends with fluorescein and fluorescein quencher, respectively. Later work also showed that fluorescence melting curves could be used to monitor hybridization (Morrison and Stols, 1993).

Hybridization Probes

Hybridization probes used in real-time PCR were developed mainly for use with the Roche LightCycler® instruments (U.S. Published Application No. 2001/6174670; U.S. Published Application No. 2000/6140054). These are sometimes referred to as FRET probes, LightCycler® probes, or dual FRET probes (Espy et al., 2006).

Hybridization probes are used in a format in which FRET is measured directly (Wilhelm and Pingoud, 2003). Each of the two probes is labeled with a respective member of a fluorescent energy transfer pair, such that upon hybridization to adjacent regions of the target DNA sequence, the excitation energy is transferred from the donor to the acceptor, and subsequent emission by the acceptor can be recorded as reporter signal (Wittwer et al., 1997). The two probes anneal to the target sequence so that the upstream probe is fluorescently labeled at its 3′ end and the downstream probe is labeled at its 5′ end. The 3′ end of the downstream probe is typically blocked by phosphorylation or some other means to prevent extension of the probe during PCR. The dye coupled to the 3′ end of the upstream probe is sufficient to prevent extension of this probe. This reporter system is different from other FRET-based detection methods (molecular beacons, TaqMan®, etc.) in that it uses FRET to generate rather than to quench the fluorescent signal (Dorak, 2006).

Typical acceptor fluorophores include the cyanine dyes (Cy3 and Cy5), 6-carboxy-4,7,2′,7′-tetrachlorofluorescein (TET), 6-carboxy-N,N,N′,N′-tetramethylrhodamine (TAMRA), and 6-carboxyrhodamine X (ROX). Donor fluorophores are usually 6-carboxyfluoroscein (FAM) (Wilhelm and Pingoud, 2003). Hybridization probes are particularly advantageous for genotyping and mismatch detection. Melting curve analysis can be performed in addition to the per-cycle monitoring of fluorescence during the PCR reaction. A slow heating of the sample after probe hybridization can provide additional qualitative information about the sequence of interest (Lay and Wittwer, 1997; Bernard et al., 1998a; Bernard et al., 1998b). Base-pair mismatches will shift the stability of a duplex, in varying amounts, depending on the mismatch type and location in the sequence (Guo et al., 1997).

Molecular Beacons

Molecular beacons, also known as hairpin probes, are stem-loop structures that open and hybridize in the presence of a complementary target sequence, typically causing an increase in fluorescence (U.S. Pat. No. 5,925,517; U.S. Published Application No. 2006/103476). Molecular beacons typically have a nucleic acid target complement sequence flanked by members of an affinity pair that, under assay conditions in the absence of target, interact with one another to form a stem duplex. Hybridization of the probes to their preselected target sequences produces a conformational change in the probes, forcing the “arms” apart and eliminating the stem duplex and thereby separating the fluorophore and quencher.

Hydrolysis Probes

Hydrolysis probes, also known as the TaqMan® assay (U.S. Pat. No. 5,210,015), are popular because they only involve a single probe per target sequence, as opposed to two probes (as in hybridization probes). This results in a cost savings per sample. The design of these probes is also less complicated than that of molecular beacons. These are typically labeled with a reporter on the 5′ end and a quencher on the 3′ end. When the reporter and quencher are fixed onto the same probe, they are forced to remain in close proximity. This proximity effectively quenches the reporter signal, even when the probe is hybridized to the target sequence. During the extension or elongation phase of the PCR reaction, a polymerase known as Taq polymerase is used because of its 5′ exonuclease activity. The polymerase uses the upstream primer as a binding site and then extends. Hydrolysis probes are cleaved during polymerase extension at their 5′ end by the 5′-exonuclease activity of Taq. When this occurs, the reporter fluorophore is released from the probe, and subsequently is no longer in close proximity to the quencher. This produces a perpetual increase in reporter signal with each extension phase as the PCR reaction continues cycling. In order to ensure maximal signal with each cycle, hydrolysis probes are designed with a Tm that is roughly 10° C. higher than the primers in the reaction.

However, the process of cleaving the 5′ end of the probe need not require amplification or extension of the target sequence (U.S. Pat. No. 5,487,972). This is accomplished by placing the probe adjacent to the upstream primer, on the target sequence. In this manner, sequential rounds of annealing and subsequent probe hydrolysis can occur, resulting in a significant amount of signal generation in the absence of polymerization. Uses of the real-time hydrolysis probe reaction are also described in U.S. Pat. Nos. 5,538,848 and 7,205,105.

Dye-Primer Based Systems

There are numerous dye-labeled primer based systems available for real-time PCR. These range in complexity from simple hairpin primer systems to more complex primer structures where the stem-loop portion of the hairpin probe is attached via a non-amplifiable linker to the specific PCR primer. These methods have the advantage that they do not require an additional intervening-labeled probe that is essential for probe-based assay systems and they also allow for multiplexing that is not possible with DNA binding dyes. However, the success of each of these methods is dependent upon careful design of the primer sequences.

Hairpin primers contain inverted repeat sequences that are separated by a sequence that is complementary to the target DNA (Nazarenko et al., 1997; Nazarenko et al., 2002; U.S. Pat. No. 5,866,336). The repeats anneal to form a hairpin structure, such that a fluorophore at the 5′ end is in close proximity to a quencher at the 3′ end, quenching the fluorescent signal. The hairpin primer is designed so that it will preferentially bind to the target DNA, rather than retain the hairpin structure. As the PCR reaction progresses, the primer anneals to the accumulating PCR product, the fluorophore and quencher become physically separated, and the level of fluorescence increases.

Invitrogen's LUX™ (Light Upon eXtension) primers are fluorogenic hairpin primers which contain a short 4-6 nucleotide extension at the 5′ end of the primer that is complementary to an internal sequence near the 3′ end and overlaps the position of a fluorophore attached near the 3′ end (Chen et al., 2004; Bustin, 2002). Basepairing between the complementary sequences forms a double-stranded stem which quenches the reporter dye that is in close proximity at the 3′ end of the primer. During PCR, the LUX™ primer is incorporated into the new DNA strand and then becomes linearized when a new complementary second strand is generated. This structural change results in an up to 10-fold increase in the fluorescent signal. These primers can be difficult to design and secondary structure must be carefully analyzed to ensure that the probe anneals preferentially to the PCR product. Design and validation services for custom LUX™ primers are available from Invitrogen.

More recently, hairpin probes have become part of the PCR primer (Bustin, 2002). In this approach, once the primer is extended, the sequence within the hairpin anneals to the newly synthesized PCR product, disrupting the hairpin and separating the fluorophore and quencher.

Scorpion® primers are bifunctional molecules in which an upstream hairpin probe sequence is covalently linked to a downstream primer sequence (U.S. Published Application No. 2001/6270967; U.S. Published Application No. 2005/0164219; Whitcombe et al., 1999). The probe contains a fluorophore at the 5′ end and a quencher at the 3′ end. In the absence of the target, the probe forms a 6-7 base stem, bringing the fluorophore and quencher in close proximity and allowing the quencher to absorb the fluorescence emitted by the fluorophore. The loop portion of the scorpion probe section consists of sequence complementary to a portion of the target sequence within 11 bases downstream from the 3′ end of the primer sequence. In the presence of the target, the probe becomes attached to the target region synthesized in the first PCR cycle. Following the second cycle of denaturation and annealing, the probe and the target hybridize. Denaturation of the hairpin loop requires less energy than the new DNA duplex produced. Thus, the scorpion probe loop sequence hybridizes to a portion of the newly produced PCR product, resulting in separation of the fluorophore from the quencher and an increase in the fluorescence emitted.

As with all dye-primer based methods, the design of Scorpion primers follows strict design considerations for secondary structure and primer sequence to ensure that a secondary reaction will not compete with the correct probing event. The primer pair should be designed to give an amplicon of approximately 100-200 bp. Ideally, the primers should have as little secondary structure as possible and should be tested for hairpin formation and secondary structures. The primer should be designed such that it will not hybridize to the probe element as this would lead to linearization and an increase in non-specific fluorescence emission. The Tm's of the two primers should be similar and the stem Tm should be 5-10° C. higher than the probe Tm. The probe sequence should be 17-27 bases in length and the probe target should be 11 bases or less from the 3′ end of the scorpion. The stem sequence should be 6 to 7 bases in length and should contain primarily cytosine and guanine. The 5′ stem sequence should begin with a cytosine as guanine may quench the fluorophore. Several oligonucleotide design software packages contain algorithms for Scorpion primer design and custom design services are available from some oligonucleotide vendors as well.

The Plexor™ system from Promega is a real-time PCR technology that has the advantage that there are no probes to design and only one PCR primer is labeled (U.S. Pat. No. 5,432,272; U.S. Published Application No. 2000/6140496; U.S. Published Application No. 2003/6617106). This technology takes advantage of the specific interaction between two modified nucleotides, isoguanine (iso-dG) and 5′-methylisocytosine (iso-dC) (Sherrill et al., 2004; Johnson et al., 2004; Moser and Prudent, 2003). Main features of this technology are that the iso-bases will only base pair with the complementary iso-base and DNA polymerase will only incorporate an iso-base when the corresponding complementary iso-base is present in the existing sequence. One PCR primer is synthesized with a fluorescently-labeled iso-dC residue as the 5′ terminal nucleotide. As amplification progresses, the labeled primer is annealed and extended, becoming incorporated in the PCR product. A quencher-labeled iso-dGTP (dabsyl-isodGTP), available as the free nucleotide in the PCR master mix, specifically base pairs with the iso-dC and becomes incorporated in the complementary PCR strand, quenching the fluorescent signal. Primer design for the Plexor system is relatively simple as compared to some of the other dye-primer systems and usually follows typical target-specific primer design considerations. A web-based Plexor Primer Design Software, available from Promega, assists in selecting the appropriate dye and quencher combinations, and provides links to oligonucleotide suppliers licensed to provide iso-base containing primers.

In places where the description above refers to particular implementations of kits and methods, it should be readily apparent that a number of modifications may be made without departing from the spirit thereof and that these implementations may be alternatively applied. The presently disclosed implementations are, therefore, to be considered in all respects as illustrative and not restrictive. All changes that come within the meaning of and range of equivalency of the disclosure, including the examples, are intended to be embraced therein.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the best mode of the methods and kits are described herein.

The disclosure is not limited to the particular methodology, protocols and materials described, as these can vary. It is also understood that the terminology used herein is for the purposes of describing particular embodiments only and is not intended to limit the scope of the present invention. Additionally, unless otherwise noted, method steps according to an aspect of the invention may be performed in any sequence possible to achieve the desired result.

### Examples

1. A method of characterizing a target molecule, the method comprising:

using one or more identifying molecules attached to the target molecule, forming two or more addressable divided sections,

wherein the one or more identifying molecules include a random or unique identifying portion, and

wherein the two or more addressable divided sections can be used to characterize the target molecule.

2. The method of example 1, wherein the two or more addressable divided sections comprise a first section and a second section.
3. The method of any of examples 1-2, further comprising a step of determining a representative first section.
4. The method of example 3, wherein the representative first section is determined based on a most-common sequence for the first section.
5. The method of example 3, wherein the representative first section is determined based on a determination of a most-common base at one or more sites of a plurality of sequences of one or more first sections.
6. The method of any of examples 1-5, further comprising a step of determining a representative second section.
7. The method of example 6, wherein the representative second section is determined based on a most-common sequence for the second section.
8. The method of example 6, wherein the representative second section is determined based on a determination of a most-common base at one or more sites of a plurality of sequences of one or more second sections.
9. The method of any of examples 6-8, wherein the representative first section and the representative second section are used to characterize the target molecule.
10. The method of any of examples 1-9 including the steps of any of examples 11-116.
11. A method of characterizing a target molecule, the method comprising the steps of:

attaching a first identifying molecule to a first end of the target molecule;

attaching a second identifying molecule to a second end of the target molecule;

forming a plurality of continuous molecules; and

dividing a first portion of the continuous molecules into addressable first sections and a second portion of the continuous molecules into addressable second sections.

12. The method of example 11, further comprising the steps of sequencing the first section to obtain a first section sequence and sequencing the second section to obtain a second section sequence.
13. The method of example 12, further comprising a step of determining a sequence of the target molecule using the first section sequence and the second section sequence.
14. The method of any of examples 12-13, further comprising a step of, using a computer, determining a sequence of the target molecule using the first section sequence and the second section sequence.
15. The method of any of examples 11-14, wherein the first identifying molecule comprises a first primer.
16. The method of example 15, wherein the first primer is a specific primer.
17. The method of example 15, wherein the first primer is a random primer.
18. The method of any of examples 11-15, wherein the first identifying molecule comprises a forward primer.
19. The method of any of examples 15-18, wherein the first or forward primer comprises a conserve region primer.
20. The method of any of examples 11-19, wherein the first identifying molecule comprises a first tag.
21. The method of example 20, wherein the first tag comprises a random molecule.
22. The method of any of examples 20-21, wherein the first tag comprises a molecule comprising one or more, two or more, three or more, four or more, or five or more bases.
23. The method of any of examples 20-22, wherein the first tag comprises a molecule comprising 2 to about 50, 2 to about 20, or 2 to about 10 bases.
24. The method of any of examples 22-23 wherein one or more of the sites of the bases or base pairs of the tag are random.
25. The method of any of examples 22-24, wherein each of base sites or base pairs is random.
26. The method of any of examples 20-25, wherein the first tag comprises a random nucleotide.
27. The method of any of examples 11-26, wherein the second identifying molecule comprises a second primer.
28. The method of example 27, wherein the second primer is a specific primer.
29. The method of example 27, wherein the second primer is a random primer.
30. The method of any of examples 11-29, wherein the second identifying molecule comprises a reverse primer.
31. The method of any of examples 27-30, wherein the second or reverse primer comprises a conserve region primer.
32. The method of any of examples 11-31, wherein the second identifying molecule comprises a second tag.
33. The method of example 32, wherein the second tag comprises a random molecule.
34. The method of any of examples 32-33, wherein the second tag comprises a molecule comprising one or more, two or more, three or more, four or more, or five or more bases.
35. The method of any of examples 32-34, wherein the second tag comprises a molecule comprising 2 to about 50, 2 to about 20, or 2 to about 10 bases.
36. The method of any of examples 34-35, wherein one or more of the sites of the bases are random.
37. The method of any of examples 34-36, wherein each of base sites or base pairs is random.
38. The method of any of examples 32-37, wherein the second tag comprises a random nucleotide.
39. The method of any of examples 13-38, wherein the step of determining comprises identifying one or more of at least a portion of the first identifying molecule and identifying at least a portion of the second identifying molecule.
40. The method of any of examples 13-38, wherein the step of determining comprises identifying one or more of a first tag of the first identifying molecule and identifying a second tag of the second identifying molecule.
41. The method of any of examples 13-40, wherein the step of determining comprises identifying a third primer.
42. The method of any of examples 13-41, wherein the step of determining comprises matching the first section having a first section single molecule tag sequence with the second section having a second section single molecule tag that is the inverse compliment of the first section single molecule tag sequence.
43. The method of any of examples 11-42, wherein the step of dividing comprises using a third identifying molecule and a fourth identifying molecule.
44. The method of example 43, wherein the third identifying molecule comprises a third primer.
45. The method of any of examples 43-44, wherein the fourth identifying molecule comprises a fourth primer.
46. The method of example 45, wherein the third primer and the fourth primer are in the opposite directions relative to one another in reference to the target molecule.
47. The method of any of examples 43-46, wherein the third primer and the first primer are inverse compliments of each other.
48. The method of any of examples 11-47, wherein the step of dividing comprises using a fifth identifying molecule and a sixth identifying molecule.
49. The method of example 48, wherein the fifth identifying molecule comprises a fifth primer.
50. The method of any of examples 48-49, wherein the sixth identifying molecule comprises a sixth primer.
51. The method of example 50, wherein the fifth primer and the sixth primer are in the opposite directions relative to one another in reference to the target molecule.
52. The method of any of examples 49-51, wherein the fifth primer and the second primer are inverse compliments of each other.
53. The method of any of examples 49-52, wherein the fifth primer comprises a random nucleotide.
54. The method of any of examples 50-53, wherein the sixth primer comprises a random nucleotide.
55. The method of any of examples 11-54, wherein the target molecule comprises a portion of a DNA strand.
56. The method of any of examples 11-55, wherein the target molecule comprises about 100-200, 125-350, 150-250, 200-300, 250-550, 300-600, 400-500, 450-550, 500-1000, 1000-5000, 5000-50000, etc. or more base pairs.
57. The method of any of examples 11-56, further additional steps of:

attaching a seventh identifying molecule to a first end of the first section;

attaching an eighth identifying molecule to a second end of the first section;

forming a plurality of continuous molecules including; and

dividing a first portion of the continuous molecules into a fifth sections and a portion of the continuous molecules into sixth sections.

58. The method of any of examples 11-56, further additional steps of:

attaching a ninth identifying molecule to a first end of the second section;

attaching an tenth identifying molecule to a second end of the second section;

forming a plurality of continuous molecules; and

dividing a first portion of the continuous molecules into a seventh sections and a portion of the continuous molecules into eighth section.

59. The method of any of examples 11-58, further comprising a step of characterizing sequence variations of the target molecule.
60. The method of any of examples 11-59, further comprising a step of determining a characteristic target molecule from a family first sections and a family of second sections.
61. The method of example 60, wherein a representative first section is selected.
62. The method of example 61, wherein the most-common first section sequence is selected as a representative of the family of first sections.
63. The method of example 60, wherein a representative second section is selected.
64. The method of example 63, wherein the most-common second section sequence is selected as a representative of the family of section sections.
65. The method of any of examples 11-64, further comprising a step of determining a probability of a base pair at one or more sites of the target molecule.
66. The method of any of examples 11-65, further comprising a step of attributing a quality score to a sequence of the target molecule.
67. The method of characterizing a target molecule of any of claims 1-66 by addressably identifying the first sections and the second section to form a characteristic target molecule.
68. A method of diagnosing disease using the method of example 67.
69. A method of determining disease risk using the method of example 67.
70. A method of characterizing a target molecule, the method comprising the steps of:

using a first amplification process, attaching a first identifying molecule to a first end of the target molecule and attaching a second identifying molecule to a second end of the target molecule;

forming a plurality of continuous molecules; and

dividing a portion of the plurality of continuous molecules into a plurality of first sections and another portion of the plurality of continuous molecules into a plurality of second sections.

71. The method of example 70, wherein the first identifying molecule comprises a first primer.
72. The method of example 71, wherein the first primer is a specific primer.
73. The method of example 71, wherein the first primer is a random primer.
74. The method of any of examples 70-71, wherein the first identifying molecule comprises a forward primer.
75. The method of example 74, wherein the forward primer comprises a conserve region primer.
76. The method of any of examples 70-75, wherein the first identifying molecule comprises a first tag.
77. The method of example 76, wherein the first tag comprises a random molecule.
78. The method of any of examples 76-77, wherein the first tag comprises a molecule comprising one or more, two or more, three or more, four or more, or five or more bases.
79. The method of any of examples 76-78, wherein the first tag comprises a molecule comprising 2 to about 50, 2 to about 20, or 2 to about 10 bases.
80. The method of any of examples 78-79, wherein one or more of the sites of the bases or base pairs are random.
81. The method of any of examples 78-80, wherein each of base sites or base pairs is random.
82. The method of any of examples 76-81, wherein the first tag comprises a random nucleotide.
83. The method of any of examples 70-82, wherein the second identifying molecule comprises a second primer.
84. The method of any of examples 70-83, wherein the second identifying molecule comprises a reverse primer.
85. The method of any of examples 83-84 wherein the reverse or second primer is a specific primer.
86. The method of any of examples 83-84, wherein the reverse or second primer is a random primer.
87. The method of example 83-86, wherein the reverse or second primer comprises a conserve region primer
88. The method of any of examples 70-87, wherein the second identifying molecule comprises a second tag.
89. The method of example 88, wherein the second tag comprises a random molecule.
90. The method of any of examples 88-89, wherein the second tag comprises a molecule comprising 2 to about 50, 2 to about 20, or 2 to about 10 bases.
91. The method off example 90, wherein one or more of the sites of the bases or base pairs are random.
92. The method of any of examples 90-91, wherein each of base sites or base pairs is random.
93. The method of any of examples 88-92, wherein the second tag comprises a random nucleotide.
94. The method of any of examples 70-93, wherein the first amplification process comprises polymerase chain reaction.
95. The method of any of examples 70-94, wherein the step of dividing comprises a second amplification process.
96. The method of example 95, wherein the second amplification process comprises polymerase chain reaction.
97. The method of any of examples 11-96, wherein the step of dividing comprises attaching a third primer.
98. The method of any of examples 11-96, wherein the step of dividing comprises attaching a fourth primer.
99. The method of any of examples 11-96, wherein the step of dividing comprises attaching a fifth primer.
100. The method of any of examples 11-96, wherein the step of dividing comprises attaching a sixth primer.
101. The method of any of examples 97-100 wherein the third primer is in a first direction.
102. The method of any of examples 98-101 wherein the fourth primer is in a second direction.
103. The method of any of examples 99-102, wherein the fifth primer is in a first direction.
104. The method of any of examples 100-103, wherein the sixth primer is in a second direction.
105. The method of any of examples 97-104, wherein the first primer and the third primer are inverse compliments of each other.
106. The method of any of examples 99-105, wherein one or both of the fifth primer and the sixth primer comprises a random nucleotide.
107. The method of any of examples 70-106, further comprising steps of sequencing the first segment and sequencing the second segment.
108. The method of any of examples 70-107, further comprising a step of determining corresponding first segments and second segments.
109. The method of any of examples 70-108, further comprising a step of reconstructing first segments and second segments.
110. The method of any of examples 70-109, further comprising a step of characterizing a sequence of the target molecule from a family of first segments and a family of second segments.
111. The method of any of examples 70-109, further comprising a step of determining a best representative of the first segments.
112. The method of example 111, wherein the step of determining a best representative of the first segments comprises determining a most common first segment sequence.
113. The method of example 111, wherein the step of determining a best representative of the first segments comprises determining a most common base at one or more sites of the first segments.
114. The method of any of examples 70-113, further comprising a step of determining a best representative of the second segments.
115. The method of example 114, wherein the step of determining a best representative of the second segments comprises determining a most common second sequence.
116. The method of example 114, wherein the step of determining a best representative of the second segments comprises determining a most common base at one or more sites of the second segments.
117. A kit for characterizing a molecule, the kit comprising:

a first identifying molecule comprising a first primer and first tag attached to the first primer; and

a second identifying molecule comprising a second primer and a second tag attached to the second primer.

118. The kit of example 117, wherein the second tag comprises a nucleotide having random sites.
119. The kit of any of examples 117-118, further comprising a third primer.
120. The kit of any of examples 117-118, further comprising a fourth primer.
121. The kit of any of examples 117-118, further comprising a fifth primer.
122. The kit of any of examples 117-118, further comprising a sixth primer.
123. The kit of any of examples 119-122, wherein the first primer and the third primer are inverse compliments of each other.
124. The kit of any of examples 120-123, wherein the fourth primer comprises a nucleotide having random sites.
125. The kit of any of examples 121-124, wherein the fourth primer comprises a nucleotide having random sites.
126. The kit of any of examples 122-125, wherein the sixth primer comprises a nucleotide having random sites.
127. The kit of any of examples 117-126, further comprising a computer readable medium, having instructions thereon to perform the steps of reconstructing the target molecule.
128. A method for preparing a sample for sequencing, the method comprising the steps of:

attaching a first identifying molecule to a first end of the target molecule;

attaching a second identifying molecule to a second end of the target molecule;

forming a plurality of continuous molecules including at least one of the first identifying molecule and the second identifying molecule; and

dividing a first portion of the plurality of continuous molecules into first sections and a second portion of the plurality of continuous molecules into second sections.

129. The method of example 127 further comprising any of the steps of examples 12-69.
130. A method for preparing a sample for sequencing, the method comprising the steps of:

using a first amplification process, attaching a first identifying molecule to a first end of a target molecule and attaching a second identifying molecule to a second end of the target molecule;

forming a plurality of continuous molecules including at least one of the first identifying molecule and the second identifying molecule; and

dividing a first portion of the plurality of continuous molecules into first sections and a second portion of the plurality of continuous molecules into second sections.

131. The method of example 129 further comprising any of the steps of examples 71-116.
132. The method of any of examples 11-116, wherein one or more of a first identifying molecule and a second identifying molecule comprises a spacer.
133. The method of example 132, wherein the spacer is between a primer and a tag.
134. The method of any of examples 11-116, wherein one or more continuous molecules include a known priming site and a random priming site.
135. The method of any of examples 11-116, wherein one or more of a first identifying molecule and a second identifying molecule comprises a barcode.
136. The method of example 135, wherein the barcode is between a primer and a tag.

What is claimed is:

1. A method of characterizing a target molecule, the method comprising:
using one or more identifying molecules attached to the target molecule, forming two or more addressable divided sections,
wherein the one or more identifying molecules include a random or unique identifying portion, and
wherein the two or more addressable divided sections can be used to characterize the target molecule.

2. The method of claim 1, wherein the two or more addressable divided sections comprise a first section and a second section.

3. The method of claim 1, further comprising a step of determining a representative first section.

4. The method of claim 3, wherein the representative first section is determined based on a most-common sequence for the first section.

5. The method of claim 3, wherein the representative first section is determined based on a determination of a most-common base at one or more sites of a plurality of sequences of one or more first sections.

6. The method of claim 1, further comprising a step of determining a representative second section.

7. The method of claim 6, wherein the representative second section is determined based on a most-common sequence for the second section.

8. The method of claim 6, wherein the representative second section is determined based on a determination of a most-common base at one or more sites of a plurality of sequences of one or more second sections.

9. A method of characterizing a target molecule, the method comprising the steps of:
attaching a first identifying molecule to a first end of the target molecule;
attaching a second identifying molecule to a second end of the target molecule;
forming a plurality of continuous molecules; and
dividing a first portion of the continuous molecules into addressable first sections and a second portion of the continuous molecules into addressable second sections.

10. The method of claim 9, further comprising the steps of sequencing the first section to obtain a first section sequence and sequencing the second section to obtain a second section sequence.

11. The method of claim 10, further comprising a step of determining a sequence of the target molecule using the first section sequence and the second section sequence.

12. The method of claim 10, further comprising a step of, using a computer, determining a sequence of the target molecule using the first section sequence and the second section sequence.

13. The method of claim 9, wherein the first identifying molecule comprises a first primer.

14. The method of claim 13, wherein the first primer is a specific primer or a random primer.

15. The method of claim 9, wherein the first identifying molecule comprises a first tag.

16. The method of claim 15, wherein the first tag comprises a random molecule.

17. The method of claim 15, wherein each of base sites or base pairs of the first tag is random.

18. The method of claim 11, wherein the step of determining comprises identifying one or more of at least a portion of the first identifying molecule and identifying at least a portion of the second identifying molecule.

19. The method of claim 11, wherein the step of determining comprises identifying one or more of a first tag of the first identifying molecule and identifying a second tag of the second identifying molecule.

20. A method of characterizing a target molecule, the method comprising the steps of:
using a first amplification process, attaching a first identifying molecule to a first end of the target molecule and attaching a second identifying molecule to a second end of the target molecule;
forming a plurality of continuous molecules; and
dividing a portion of the plurality of continuous molecules into a plurality of first sections and another portion of the plurality of continuous molecules into a plurality of second sections.


SEQUENCE LISTING (20 sequences)

SEQ ID NO: 1 - DNA, 20 bp, Artificial sequence
  Synthetic primer sequence
agagtttgatcctggctcag

SEQ ID NO: 2 - DNA, 15 bp, Artificial sequence
  Synthetic primer sequence
ctgctgcctnccgta

SEQ ID NO: 3 - DNA, 30 bp, Artificial sequence
  Synthetic adapter sequence
ccatctcatccctgcgtgtctccgactcag

SEQ ID NO: 4 - DNA, 10 bp, Artificial sequence
  Synthetic barcode sequence
cagatccatc

SEQ ID NO: 5 - DNA, 20 bp, Artificial sequence
  Synthetic primer sequence
ctgagccaggatcaaactct

SEQ ID NO: 6 - DNA, 23 bp, Artificial sequence
  Synthetic adapter sequence
cctctctatgggcagtcggtgat

SEQ ID NO: 7 - DNA, 20 bp, Artificial sequence
  Synthetic primer sequence
ttactcacccgtncgccnct

SEQ ID NO: 8 - DNA, 30 bp, Artificial sequence; identical to SEQ ID NO: 3
  Synthetic adapter sequence

SEQ ID NO: 9 - DNA, 10 bp, Artificial sequence
  Synthetic barcode sequence
cagatccatc

SEQ ID NO: 10 - DNA, 15 bp, Artificial sequence
  Synthetic primer sequence
tacggnaggcagcag

SEQ ID NO: 11 - DNA, 23 bp, Artificial sequence
  Synthetic adapter sequence
cctctctatgggcagtcggtgat

SEQ ID NO: 12 - DNA, 20 bp, Artificial sequence
  Synthetic primer sequence
ttactcacccgtncgccnct

SEQ ID NO: 13 - DNA, 13 bp, Artificial sequence
  Synthetic amplification priming site sequence
gctcggaccgtgg

SEQ ID NO: 14 - DNA, 15 bp, Artificial sequence
  Synthetic artificial random primer sequence
cctgggccctggccg

SEQ ID NO: 15 - DNA, 13 bp, Artificial sequence
  Synthetic amplification priming site sequence
gctcggaccgtgg

SEQ ID NO: 16 - DNA, 15 bp, Artificial sequence
  Synthetic artificial priming site sequence
ggccacggacccggc

SEQ ID NO: 17 - DNA, 30 bp, Artificial sequence; identical to SEQ ID NO: 3
  Synthetic adapter sequence

SEQ ID NO: 18 - DNA, 15 bp, Artificial sequence
  Synthetic primer sequence
gccgggtccgtggcc

SEQ ID NO: 19 - DNA, 23 bp, Artificial sequence
  Synthetic adapter sequence
cctctctatgggcagtcggtgat

SEQ ID NO: 20 - DNA, 15 bp, Artificial sequence
  Synthetic primer sequence
nnnnnnnnnnnnnnn